(19) United States
(12) Patent Application Publication
(10) Pub. No.: US 2023/0151083 A1
(43) Pub. Date: May 18, 2023

(54) ANTI-PHF-TAU ANTIBODIES AND USES THEREOF

(71) Applicant: Janssen Biotech, Inc., Horsham, PA (US)

(72) Inventors: Kristof Van Kolen, Haacht (BE); Marc Mercken, Turnhout (BE); Rupesh Nanjunda, Hatfield, PA (US); Sanjaya Singh, Blue Bell, PA (US); Sherry La Porte, Horsham, PA (US); Jinquan Luo, Malvern, PA (US); Pharavee Jaiprasart, North Wales, PA (US); Sathya Venkataramani, Blue Bell, PA (US); Rajkumar Ganesan, Thousand Oaks, CA (US)

(21) Appl. No.: 17/917,916

(22) PCT Filed: Apr. 7, 2021
(86) PCT No.: PCT/IB2021/052890

§ 371 Date: Oct. 7, 2022

Related U.S. Application Data
(60) Provisional application No. 63/007,118, filed on Apr. 8, 2020.
(60) Provisional application No. 63/026,387, filed on May 18, 2020.

(51) Int. Cl. C07K 16/18; G01N 33/68; A61P 25/28; C12N 15/63

(52) U.S. Cl. CPC C07K 16/18; G01N 33/6896; A61P 25/28; C12N 15/63; C07K 2317/56

(57) ABSTRACT

Monoclonal anti-PHF-tau antibodies and antigen-binding fragments thereof are described. Also described are nucleic acids encoding the antibodies, compositions comprising the antibodies, methods of producing the antibodies and using the antibodies for treating or preventing conditions such as tauopathies.

### CROSS-REFERENCE TO RELATED APPLICATIONS

This application claims the benefit of priority of U.S. Application Ser. No. 63/007,118, filed on Apr. 8, 2020, and U.S. Application Ser. No. 63/026,387, filed on May 18, 2020.

### FIELD OF THE INVENTION

The invention relates to anti-PHF-tau antibodies, nucleic acids and expression vectors encoding the antibodies, recombinant cells containing the vectors, and compositions comprising the antibodies. Methods of making the antibodies, methods of using the antibodies to treat conditions including tauopathies, and methods of using the antibodies to diagnose diseases such as tauopathies are also provided.

### REFERENCE TO SEQUENCE LISTING SUBMITTED ELECTRONICALLY

This application contains a sequence listing, which is submitted electronically via EFS-Web as an ASCII formatted sequence listing with a file name “JBI6174WOPCT1_SL” and a creation date of Apr. 2, 2021 and having a size of 169,953 bytes. The sequence listing submitted via EFS-Web is part of the specification and is herein incorporated by reference in its entirety.

### BACKGROUND OF THE INVENTION

Alzheimer's Disease (AD) is a degenerative brain disorder characterized clinically by progressive loss of memory, cognition, reasoning, judgment and emotional stability that gradually leads to profound mental deterioration and ultimately death. AD is a very common cause of progressive mental failure (dementia) in aged humans and is believed to represent the fourth most common medical cause of death in the United States. AD has been observed in ethnic groups worldwide and presents a major present and future public health problem.

The brains of individuals with AD exhibit characteristic lesions termed senile (or amyloid) plaques, amyloid angiopathy (amyloid deposits in blood vessels) and neurofibrillary tangles. Large numbers of these lesions, particularly amyloid plaques and neurofibrillary tangles of paired helical filaments, are generally found in several areas of the human brain important for memory and cognitive function in patients with AD.

The current AD treatment landscape includes only therapies approved to treat cognitive symptoms in patients with dementia. There are no approved therapies that modify or slow the progression of AD. Potential disease modifiers include Eli Lilly's humanized anti-An monoclonal Solanezumab for patients with mild AD and Merck's small molecule BACE inhibitor Verubecestat for patients with mild-to-moderate AD. These therapies, and most other potential disease modifiers that may launch in the next decade, target A_{β} (the principal component of the amyloid plaques that are one of the two “hallmark” pathological signs of AD).

Neurofibrillary tangles, the second hallmark pathological sign of AD, are primarily composed of aggregates of hyper-phosphorylated tau protein. The main physiological function of tau is microtubule polymerization and stabilization. The binding of tau to microtubules takes place by ionic interactions between positive charges in the microtubule binding region of tau and negative charges on the microtubule lattice (Butner and Kirschner, J Cell Biol. 115(3):717-30, 1991). Tau protein contains 85 possible phosphorylation sites and phosphorylation at many of these sites interferes with the primary function of tau. Tau that is bound to the axonal microtubule lattice is in a hypo-phosphorylation state, while aggregated tau in AD is hyper-phosphorylated, providing unique epitopes that are distinct from the physiologically active pool of tau.

A tauopathy transmission and spreading hypothesis has been described and is based on the Braak stages of tauopathy progression in the human brain and tauopathy spreading after tau aggregate injections in preclinical tau models (Frost et al., J Biol Chem. 284:12845-52, 2009; Clavaguera et al., Nat Cell Biol. 11:909-13, 2009).

Developing therapeutics preventing or clearing tau aggregation has been of interest for many years and candidate drugs, including anti-aggregation compounds and kinase inhibitors, have entered in clinical testing (Brunden et al., Nat Rev Drug Discov. 8:783-93, 2009). Multiple studies have been published that show the beneficial therapeutic effects of both active and passive tau immunization in transgenic mouse models (Chai et al., J Biol Chem. 286:34457-67, 2011; Boutajangout et al., J Neurochem. 118:658-67, 2011; Boutajangout et al., J Neurosci. 30:16559-66, 2010; Asuni et al., J Neurosci. 27:9115-29, 2007). Activity has been reported with both phospho-directed and non-phospho-directed antibodies (Schroeder et al., J Neuroimmune Pharmacol. 11(1):9-25, 2016).

Despite the progress, there remains a need for effective therapeutics that prevent tau aggregation and tauopathy progression to treat tauopathies such as AD and other neurodegenerative diseases.

### BRIEF SUMMARY OF THE INVENTION

The invention satisfies this need by providing anti-PHF-tau antibodies or antigen-binding fragments thereof that have high binding affinity towards paired helical filament (PHF)-tau and are selective for phosphorylated tau. Antibodies of the invention were generated by human framework adaptation (HFA) of mouse PHF-tau-specific antibodies. It is thought that the selectivity of the antibodies for phosphorylated tau allows for efficacy against pathogenic tau without interfering with normal tau function. The invention also provides nucleic acids encoding the antibodies, compositions comprising the antibodies, and methods of making and using the antibodies. Anti-PHF-tau antibodies or antigen-binding fragments thereof of the invention inhibit tau seeds, as measured by cellular assays using tau seeds derived from HEK cell lysates or from spinal cord lysates from mutant tau transgenic mice. In addition, a chimeric antibody with variable regions of anti-PHF-tau antibodies of the invention and mouse Ig constant regions, such as mouse IgG2a constant regions, blocked seeding activity in an in vivo mutant tau transgenic mouse model.

The progression of tauopathy in an AD brain follows distinct spatial spreading patterns. It has been shown in preclinical models that extracellular phospho-tau seeds can induce tauopathy in neurons (Clavaguera et al., PNAS 110(23):9535-40, 2013). It is therefore believed that tauopathy can spread in a prion-like fashion from one brain region to the next. This spreading process would involve an externalization of tau seeds that can be taken up by nearby neurons and induce further tauopathy. While not wishing to be bound by theory, it is thought that anti-PHF-tau antibodies or antigen-binding fragments thereof of the invention prevent tau aggregation or the spreading of tauopathy in the brain by interacting with phospho-tau seeds.

In one general aspect, the invention relates to an isolated monoclonal antibody or an antigen-binding fragment thereof that binds PHF-tau. In a specific embodiment, the antibody is a humanized monoclonal antibody.

According to a particular aspect, the invention relates to an isolated monoclonal antibody or antigen-binding fragment thereof comprising a heavy chain variable region having a polypeptide sequence selected from SEQ ID NO: 71, 45, 51, 57, 63, 69, 39, 41, 43, 47, 49, 53, 55, 59, 61, 65, or 67, or a light chain variable region having a polypeptide sequence selected from SEQ ID NO: 72, 46, 52, 58, 64, 70, 40, 42, 44, 48, 50, 54, 56, 62, 66, or 68, wherein the monoclonal antibody or antigen-binding fragment thereof specifically binds paired helical filament (PHF)-tau, preferably human PHF-tau.

According to another particular aspect, the isolated monoclonal antibody or antigen-binding fragment thereof comprises:
a. a heavy chain variable region having the polypeptide sequence of SEQ ID NO: 71, and a light chain variable region having the polypeptide sequence of SEQ ID NO: 72;
b. a heavy chain variable region having the polypeptide sequence of SEQ ID NO:45, and a light chain variable region having the polypeptide sequence of SEQ ID NO:46;
c. a heavy chain variable region having the polypeptide sequence of SEQ ID NO: 51, and a light chain variable region having the polypeptide sequence of SEQ ID NO: 52;
d. a heavy chain variable region having the polypeptide sequence of SEQ ID NO: 57, and a light chain variable region having the polypeptide sequence of SEQ ID NO: 58;
e. a heavy chain variable region having the polypeptide sequence of SEQ ID NO: 63, and a light chain variable region having the polypeptide sequence of SEQ ID NO: 64;
f. a heavy chain variable region having the polypeptide sequence of SEQ ID NO: 69, and a light chain variable region having the polypeptide sequence of SEQ ID NO: 70;
g. a heavy chain variable region having the polypeptide sequence of SEQ ID NO: 39, and a light chain variable region having the polypeptide sequence of SEQ ID NO: 40;
h. a heavy chain variable region having the polypeptide sequence of SEQ ID NO: 41, and a light chain variable region having the polypeptide sequence of SEQ ID NO: 42;
i. a heavy chain variable region having the polypeptide sequence of SEQ ID NO: 43, and a light chain variable region having the polypeptide sequence of SEQ ID NO: 44;
j. a heavy chain variable region having the polypeptide sequence of SEQ ID NO: 47, and a light chain variable region having the polypeptide sequence of SEQ ID NO: 48;
k. a heavy chain variable region having the polypeptide sequence of SEQ ID NO: 49, and a light chain variable region having the polypeptide sequence of SEQ ID NO: 50;
l. a heavy chain variable region having the polypeptide sequence of SEQ ID NO: 53, and a light chain variable region having the polypeptide sequence of SEQ ID NO: 54;
m. a heavy chain variable region having the polypeptide sequence of SEQ ID NO: 55, and a light chain variable region having the polypeptide sequence of SEQ ID NO: 56;
n. a heavy chain variable region having the polypeptide sequence of SEQ ID NO: 59, and a light chain variable region having the polypeptide sequence of SEQ ID NO: 60;
o. a heavy chain variable region having the polypeptide sequence of SEQ ID NO: 61, and a light chain variable region having the polypeptide sequence of SEQ ID NO: 62;
p. a heavy chain variable region having the polypeptide sequence of SEQ ID NO: 65, and a light chain variable region having the polypeptide sequence of SEQ ID NO: 66; or
q. a heavy chain variable region having the polypeptide sequence of SEQ ID NO: 67, and a light chain variable region having the polypeptide sequence of SEQ ID NO: 68.

According to another particular aspect, the isolated monoclonal antibody or antigen-binding fragment thereof comprises:
a. a heavy chain having the polypeptide sequence of SEQ ID NO: 37, and a light chain having the polypeptide sequence of SEQ ID NO: 38;
b. a heavy chain having the polypeptide sequence of SEQ ID NO: 11, and a light chain having the polypeptide sequence of SEQ ID NO: 12;
c. a heavy chain having the polypeptide sequence of SEQ ID NO: 17, and a light chain having the polypeptide sequence of SEQ ID NO: 18;
d. a heavy chain having the polypeptide sequence of SEQ ID NO: 23, and a light chain having the polypeptide sequence of SEQ ID NO: 24;
e. a heavy chain having the polypeptide sequence of SEQ ID NO: 29, and a light chain having the polypeptide sequence of SEQ ID NO: 30;
f. a heavy chain having the polypeptide sequence of SEQ ID NO: 35, and a light chain having the polypeptide sequence of SEQ ID NO: 36;
g. a heavy chain having the polypeptide sequence of SEQ ID NO: 5, and a light chain having the polypeptide sequence of SEQ ID NO: 6;
h. a heavy chain having the polypeptide sequence of SEQ ID NO: 7, and a light chain having the polypeptide sequence of SEQ ID NO: 8;
i. a heavy chain having the polypeptide sequence of SEQ ID NO: 9, and a light chain having the polypeptide sequence of SEQ ID NO: 10;
j. a heavy chain having the polypeptide sequence of SEQ ID NO: 13, and a light chain having the polypeptide sequence of SEQ ID NO: 14;
k. a heavy chain having the polypeptide sequence of SEQ ID NO: 15, and a light chain having the polypeptide sequence of SEQ ID NO: 16;
l. a heavy chain having the polypeptide sequence of SEQ ID NO: 19, and a light chain having the polypeptide sequence of SEQ ID NO: 20;
m. a heavy chain having the polypeptide sequence of SEQ ID NO: 21, and a light chain having the polypeptide sequence of SEQ ID NO: 22;
n. a heavy chain having the polypeptide sequence of SEQ ID NO: 25, and a light chain having the polypeptide sequence of SEQ ID NO: 26;
o. a heavy chain having the polypeptide sequence of SEQ ID NO: 27, and a light chain having the polypeptide sequence of SEQ ID NO: 28;
p. a heavy chain having the polypeptide sequence of SEQ ID NO: 31, and a light chain having the polypeptide sequence of SEQ ID NO: 32; or
q. a heavy chain having the polypeptide sequence of SEQ ID NO: 33, and a light chain having the polypeptide sequence of SEQ ID NO: 34.

In another general aspect, the invention relates to an isolated nucleic acid encoding a monoclonal antibody or antigen-binding fragment thereof of the invention.

In another general aspect, the invention relates to a vector comprising an isolated nucleic acid encoding a monoclonal antibody or antigen-binding fragment thereof of the invention.

In another general aspect, the invention relates to a host cell comprising an isolated nucleic acid encoding a monoclonal antibody or antigen-binding fragment thereof of the invention.

In another general aspect, the invention relates to a pharmaceutical composition comprising an isolated monoclonal antibody or antigen-binding fragment thereof of the invention and a pharmaceutically acceptable carrier.

In another general aspect, the invention relates to a method of reducing pathological tau aggregation or spreading of tauopathy in a subject in need thereof, comprising administering to the subject a pharmaceutical composition of the invention.

In another general aspect, the invention relates to a method of treating a tauopathy in a subject in need thereof, comprising administering to the subject a pharmaceutical composition of the invention. The tauopathy includes, but is not limited to, one or more selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

In another general aspect, the invention relates to a method of producing a monoclonal antibody or antigen-binding fragment thereof of the invention, comprising culturing a cell comprising a nucleic acid encoding the monoclonal antibody or antigen-binding fragment thereof under conditions to produce the monoclonal antibody or antigen-binding fragment thereof and recovering the monoclonal antibody or antigen-binding fragment thereof from the cell or cell culture.

In another general aspect, the invention relates to a method of producing a pharmaceutical composition comprising a monoclonal antibody or antigen-binding fragment thereof of the invention, comprising combining the monoclonal antibody or antigen-binding fragment thereof with a pharmaceutically acceptable carrier to obtain the pharmaceutical composition.

In another general aspect, the invention relates to a method of detecting the presence of phosphorylated PHF-tau in a subject or a method of diagnosing a tauopathy in a subject by detecting the presence of PHF-tau in the subject using a monoclonal antibody or antigen-binding fragment thereof of the invention.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 shows a schematic of the humanization process.

FIGS. 2A-2AI show surface plasmon resonance profiles to evaluate binding to NPT-6 phospho peptide of rehumanized PT3 Fabs derived from supernatants of transfected E coli cells.

FIG. 3 shows a graph demonstrating the binding of mAbs to the pT217+ peptide in a direct ELISA experiment. Binding profile of the PT3 parent and from PT1B296 (i.e. the variant obtained by a previous humanization (U.S. Pat. Publication No. 2018/0265575)) were added for comparison.

FIGS. 4A-4I show representative SPR binding data of mAbs and of Fab fragments of selected rehumanized variants, PT3, and PT1B296 to NPT6-peptide.

FIGS. 5A-5F show representative SPR binding data of Fab fragments of selected rehumanized variants, PT3, and PT1B296 to PHF.

FIGS. 6A-6H show species cross-reactivity data from selected rehumanized variants, PT3, and PT1B296 determined by Western blot analysis on brain homogenates from Mouse WT (Lane 1), Mouse KO (Lane 2), Rat (Lane 3), Dog (Lane 4), minipig (Lane 5), marmoset (Lane 6) Cynomolgous monkey (Lane 7), and human (heat-stable extract from non-AD brain (Lane 8); and PHF derived from AD brain (stage Braak VI) (Lane 9)). Detection with HRPO labelled PT9 (Vandermeeren et al., J. Alzheimers Dis. 65(1):265-81 (2018)) was performed to estimate the total tau signals in the different extracts. FIG. 6A: PT3 hIgGl; FIG. 6B: PT1B296; FIG. 6C: PT1B844; FIG. 6D: PT1B847; FIG. 6E: PT1B856; FIG. 6F: PT1B850; FIG. 6G: PT1B333; and FIG. 6H: PT9-HRPO.

FIGS. 7A-7C show binding data for mAbs from selected rehumanized variants, PT3, and PT1B296 on AD and non-AD brain cryosections.

FIGS. 8A-8B show efficacy of selected rehumanized variants, PT3, and PT1B296 in a cellular model using the FRET assay as described in FIG. 8A. The graph in FIG. 8B indicates % remaining seeding in function of increasing antibody concentrations added to the AD brain-derived tau seeds.

FIGS. 9A-9D show efficacy of PT1B844, PT1B296, and PT3 in the ePHF injection model (see, e.g., U.S. Pat. Publication No. 2018/0265575) after co-administration.

FIGS. 10A-10B show the efficacy of rehumanized variants, PT1B296, and PT3 in an ex vivo model of pT217+epitope engagement (see, e.g., U.S. Pat. Publication No. 2019/0271710).

FIG. 11 shows plasma and CSF PK for PT1B844/PT1B916 after a single dose in Cynomolgous Monkey.

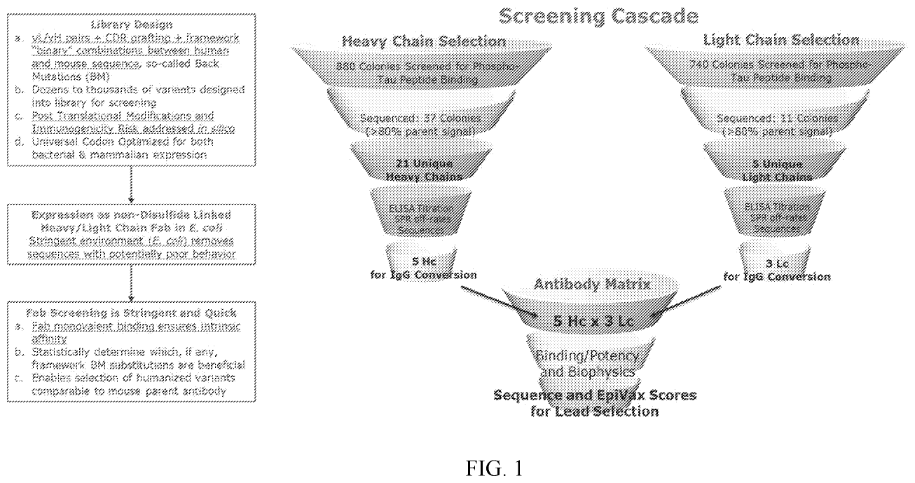

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Definitions

Unless defined otherwise, all technical and scientific terms used herein have the same meaning commonly understood to one of ordinary skill in the art to which this invention pertains. Otherwise, certain terms used herein have the meanings as set in the specification. All patents, published patent applications, and publications cited herein are incorporated by reference as if set forth fully herein.

It must be noted that as used herein and in the appended claims, the singular forms “a,” “an,” and “the” include plural reference unless the context clearly dictates otherwise.

Unless otherwise stated, any numerical value, such as a concentration or a concentration range described herein, is to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids.

As used herein, the term “antibody” or “immunoglobulin” is used in a broad sense and includes immunoglobulin or antibody molecules including polyclonal antibodies, monoclonal antibodies including murine, human, human-adapted, humanized and chimeric monoclonal antibodies and antibody fragments.

In general, antibodies are proteins or peptide chains that exhibit binding specificity to a specific antigen. Antibody structures are well known. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibodies of the invention include those that have variations in their Fc region such that they have altered properties as compared to wild type Fc regions including, but not limited to, extended half-life, reduced or increased ADCC or CDC and silenced Fc effector functions. Antibody light chains of any vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from mouse antibodies or human antibodies.

In addition to the heavy and light chain constant domains, antibodies contain light and heavy chain variable regions. An immunoglobulin light or heavy chain variable region consists of a “framework” region interrupted by “antigen-binding sites.” The antigen-binding sites are defined using various terms and numbering schemes as follows:
(i) Kabat: “Complementarity Determining Regions” or “CDRs” are based on sequence variability (Wu and Kabat, J ExpMed. 132:211-50, 1970). Generally, the antigen-binding site has three CDRs in each variable region (e.g., HCDR1, HCDR2 and HCDR3 in the heavy chain variable region (VH) and LCDR1, LCDR2 and LCDR3 in the light chain variable region (VL));
(ii) Chothia: The term “hypervariable region,” “HVR” or “HV” refers to the regions of an antibody variable domain which are hypervariable in structure as defined by Chothia and Lesk (Chothia and Lesk, J Mol Biol. 196:901-17, 1987). Generally, the antigen-binding site has three hypervariable regions in each VH (H1, H2, H3) and VL (L1, L2, L3). Numbering systems as well as annotation of CDRs and HVs have been revised by Abhinandan and Martin (Abhinandan and Martin, Mol Immunol. 45:3832-9, 2008);
(iii) IMGT: Another definition of the regions that form the antigen-binding site has been proposed by Lefranc (Lefranc et al., Dev Comp Immunol. 27:55-77, 2003) based on the comparison of V domains from immunoglobulins and T-cell receptors. The International ImMunoGeneTics (IMGT) database provides a standardized numbering and definition of these regions. The correspondence between CDRs, HVs and IMGT delineations is described in Lefranc et al., 2003, Id.;
(iv) AbM: A compromise between Kabat and Chothia numbering schemes is the AbM numbering convention described by Martin (Martin ACR (2010) Antibody Engineering, eds Kontermann R, Dubel S (Springer-Verlag, Berlin), Vol 2, pp 33-51).
(v) The antigen-binding site can also be delineated based on “Specificity Determining Residue Usage” (SDRU) (Almagro, Mol Recognit. 17:132-43, 2004), where SDR, refers to amino acid residues of an immunoglobulin that are directly involved in antigen contact.

“Framework” or “framework sequence” is the remaining sequences within the variable region of an antibody other than those defined to be antigen-binding site sequences. Because the exact definition of an antigen-binding site can be determined by various delineations as described above, the exact framework sequence depends on the definition of the antigen-binding site. The framework regions (FRs) are the more highly conserved portions of variable domains. The variable domains of native heavy and light chains each comprise four FRs (FR1, FR2, FR3 and FR4, respectively) which generally adopt a beta-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817, 1992; Tramontano et al., J. Mol. Biol. 215:175-182, 1990). Despite their high sequence variability, five of the six loops adopt just a small repertoire of main-chain conformations, called “canonical structures.” These conformations are first of all determined by the length of the loops and secondly by the presence of key residues at certain positions in the loops and in the framework regions that determine the conformation through their packing, hydrogen bonding or the ability to assume unusual main-chain conformations.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the constant region of the heavy chain. According to other particular embodiments, the antigen-binding fragment comprises Fab and F(ab′).

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “epitope” refers to a site on an antigen to which an immunoglobulin, antibody, or antigen-binding fragment thereof, specifically binds. Epitopes can be formed both from contiguous amino acids or from noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

As used herein, the term “tau” or “tau protein” refers to an abundant central and peripheral nervous system protein having multiple isoforms. In the human central nervous system (CNS), six major tau isoforms ranging in size from 352 to 441 amino acids in length exist due to alternative splicing (Hanger et al., Trends Mol Med. 15:112-9, 2009). The isoforms differ from each other by the regulated inclusion of 0-2 N-terminal inserts, and 3 or 4 tandemly arranged microtubule-binding repeats, and are referred to as ON3R (SEQ ID NO: 73), 1N3R (SEQ ID NO: 74), 2N3R (SEQ ID NO: 75), ON4R (SEQ ID NO: 76), 1N4R (SEQ ID NO: 77) and 2N4R (SEQ ID NO: 78). As used herein, the term “control tau” refers to the tau isoform of SEQ ID NO: 78 that is devoid of phosphorylation and other post-translational modifications. As used herein, the term “tau” includes proteins comprising mutations, e.g., point mutations, fragments, insertions, deletions and splice variants of full-length wild type tau. The term “tau” also encompasses post-translational modifications of the tau amino acid sequence. Post-translational modifications include, but are not limited to, phosphorylation.

Tau binds microtubules and regulates transport of cargo through cells, a process that can be modulated by tau phosphorylation. In AD and related disorders, abnormal phosphorylation of tau is prevalent and thought to precede and/or trigger aggregation of tau into fibrils, termed paired helical filaments (PHF). The major constituent of PHF is hyper-phosphorylated tau. As used herein, the term “paired helical filament-tau” or “PHF-tau” refers to tau aggregates in paired helical filaments. Two major regions in PHF structure are evident in electron microscopy, the fuzzy coat and the core filament; the fuzzy coat being sensitive to proteolysis and located outside of the filaments, and the protease-resistant core of filaments forming the backbone of PHFs (Wischik et al. Proc Natl Acad Sci USA. 85:4884-8, 1988).

An “isolated humanized antibody that binds PHF-tau” or an “isolated humanized anti-PHF-tau antibody,” as used herein, is intended to refer to a humanized anti-PHF-tau antibody which is substantially free of other antibodies having different antigenic specificities (for instance, an isolated humanized anti-PHF-tau antibody is substantially free of antibodies that specifically bind antigens other than PHF-tau). An isolated humanized anti-PHF-tau antibody can, however, have cross-reactivity to other related antigens, for instance from other species (such as PHF-tau species homologs).

As used herein, the term “specifically binds” or “specific binding” refers to the ability of an anti-PHF-tau antibody of the invention to bind to a predetermined target with a dissociation constant (K_{D}) of about 1×10⁻⁶ M or tighter, for example, about 1×10⁻⁷ M or less, about 1×10⁻⁸ M or less, about 1×10⁻⁹M or less, about 1×10⁻¹⁰ M or less, about 1×10⁻¹¹ M or less, about 1×10⁻¹² M or less, or about 1×10⁻¹³ M or less. The K_{D} is obtained from the ratio of Kd to Ka (i.e., Kd/Ka) and is expressed as a molar concentration (M). K_{D} values for antibodies can be determined using methods in the art in view of the present disclosure. For example, the K_{D} value of an anti-PHF-tau antibody can be determined by using surface plasmon resonance, such as by using a biosensor system, e.g., a Biacore® system, a Proteon instrument (BioRad) , a KinExA instrument (Sapidyne), ELISA or competitive binding assays known to those skilled in the art. Typically, an anti-PHF-tau antibody binds to a predetermined target (i.e. PHF-tau) with a K_{D} that is at least ten-fold less than its K_{D} for a nonspecific target as measured by surface plasmon resonance using, for example, a ProteOn Instrument (BioRad). The anti-PHF-tau antibodies that specifically bind to PHF-tau can, however, have cross-reactivity to other related targets, for example, to the same predetermined target from other species (homologs).

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression,” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed humanized antibody or antigen-binding fragment thereof that binds PHF-tau can be within the cytoplasm of a host cell, into the extracellular milieu such as the growth medium of a cell culture or anchored to the cell membrane.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody pharmaceutical composition can be used in the invention.

As used herein, the term “subject” refers to an animal, and preferably a mammal. According to particular embodiments, the subject is a mammal including a non-primate (e.g., a camel, donkey, zebra, cow, pig, horse, goat, sheep, cat, dog, rat, rabbit, guinea pig or mouse) or a primate (e.g., a monkey, chimpanzee, or human). In particular embodiments, the subject is a human.

As used herein, the term “therapeutically effective amount” refers to an amount of an active ingredient or component that elicits the desired biological or medicinal response in a subject. A therapeutically effective amount can be determined empirically and in a routine manner, in relation to the stated purpose. For example, in vitro assays can optionally be employed to help identify optimal dosage ranges. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the route of administration, and the severity of disease, and should be decided according to the judgment of the practitioner and each patient's circumstances. Effective doses can be extrapolated from dose-response curves derived from in vitro or animal model test systems.

As used herein, the terms “treat,” “treating,” and “treatment” are all intended to refer to an amelioration or reversal of at least one measurable physical parameter related to a tauopathy which is not necessarily discernible in the subject but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the tauopathy. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

As used herein a “tauopathy” encompasses any neurodegenerative disease that involves the pathological aggregation of tau within the brain. In addition to familial and sporadic AD, other exemplary tauopathies are frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, and chronic traumatic encephalopathy, such as dementia pugulistica (boxing disease) (Morris et al., Neuron, 70:410-26, 2011).

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

Anti-PHF-tau Antibodies

In one general aspect, the invention relates to isolated monoclonal antibodies or antigen-binding fragments thereof that bind PHF-tau. Such anti-PHF-tau antibodies can have the properties of binding a phosphorylated epitope on PHF-tau or binding to a non-phosphorylated epitope on PHF-tau. Anti-PHF-tau antibodies can be useful as therapeutics, and as research or diagnostic reagents to detect PHF-tau in biological samples, for example in tissues or cells.

According to a particular aspect, the invention relates to an isolated humanized antibody or an antigen-binding fragment thereof that binds to a phosphorylated tau protein at an epitope in the proline rich domain of the tau protein. In a more particular aspect, the invention relates to an isolated humanized antibody or an antigen-binding fragment thereof that binds to a phosphorylated tau protein at an epitope comprising phosphorylated T212 and/or T217 residues.

Humanized antibodies have variable region framework residues substantially from a human antibody (termed an acceptor antibody) and complementarity determining regions substantially from a non-human antibody (i.e., mouse-antibody), (referred to as the donor immunoglobulin). See Queen et al., Proc. Natl. Acad. Sci. USA. 86:10029-10033, 1989, WO 90/07861, U.S. Pat. Nos. 5,693,762, 5,693,761, 5,585,089, 5,530,101, and U.S. Pat. No. 5,225,539. The constant region(s), if present, is/are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. See, e.g., International Patent Publication No. WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid: (1) noncovalently binds antigen directly, (2) is adjacent to a CDR region, (3) otherwise interacts with a CDR region (e.g. is within about 6 angstroms of a CDR region), or (4) participates in the VL-VH interface.

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

Antibody humanization can be accomplished using well known methods, such as specificity determining residues resurfacing (SDRR) (US2010/0261620), resurfacing (Padlan et al., Mol. Immunol. 28:489-98, 1991), super humanization (WO 04/006955) and human string content optimization (U.S. Pat. No. 7,657,380). Human framework sequences useful for grafting or humanization can be selected from relevant databases by those skilled in the art. The selected frameworks can further be modified to preserve or enhance binding affinity by techniques such as those disclosed in Queen et al., 1989, Id. According to particular embodiments, methods for humanizing anti-PHF-tau antibodies from mouse parental antibodies include those described in the Examples below.

Antibodies of the present invention can be produced by a variety of techniques, for example by the hybridoma method (Kohler and Milstein, Nature. 256:495-7, 1975). Chimeric monoclonal antibodies containing a light chain and heavy chain variable region derived from a donor antibody (typically murine) in association with light and heavy chain constant regions derived from an acceptor antibody (typically another mammalian species such as human) can be prepared by a method disclosed in U.S. Pat. No. 4,816,567. CDR-grafted monoclonal antibodies having CDRs derived from a non-human donor immunoglobulin (typically murine) and the remaining immunoglobulin-derived parts of the molecule being derived from one or more human immunoglobulins can be prepared by techniques known to those skilled in the art such as that disclosed in US5225539. Fully human monoclonal antibodies lacking any non-human sequences can be prepared from human immunoglobulin transgenic mice by techniques referenced in Lonberg et al., Nature. 368:856-9, 1994; Fishwild et al., Nat Biotechnol. 14:845-51, 1996; and Mendez et al., Nat Genet. 15:146-56, 1997. Human monoclonal antibodies can also be prepared and optimized from phage display libraries (see, e.g., Knappik et al., J Mol Biol. 296:57-86, 2000; Krebs et al., J Immunol Methods. 254:67-84, 2001; Shi et al., J Mol Biol. 397:385-96, 2010).

Provided herein are isolated monoclonal antibodies or antigen-binding fragments thereof comprising a heavy chain variable region having a polypeptide sequence selected from SEQ ID NO: 71, 45, 51, 57, 63, 69, 39, 41, 43, 47, 49, 53, 55, 59, 61, 65, or 67, or a light chain variable region having a polypeptide sequence selected from SEQ ID NO: 72, 46, 52, 58, 64, 70, 40, 42, 44, 48, 50, 54, 56, 62, 66, or 68, wherein the monoclonal antibody or antigen-binding fragment thereof specifically binds paired helical filament (PHF)-tau, preferably human PHF-tau.

According to a particular aspect, the invention relates to isolated monoclonal antibodies or antigen-binding fragments thereof comprising:
a. a heavy chain variable region having the polypeptide sequence of SEQ ID NO: 71, and a light chain variable region having the polypeptide sequence of SEQ ID NO: 72;
b. a heavy chain variable region having the polypeptide sequence of SEQ ID NO:45, and a light chain variable region having the polypeptide sequence of SEQ ID NO:46;
c. a heavy chain variable region having the polypeptide sequence of SEQ ID NO: 51, and a light chain variable region having the polypeptide sequence of SEQ ID NO: 52;
d. a heavy chain variable region having the polypeptide sequence of SEQ ID NO: 57, and a light chain variable region having the polypeptide sequence of SEQ ID NO: 58;
e. a heavy chain variable region having the polypeptide sequence of SEQ ID NO: 63, and a light chain variable region having the polypeptide sequence of SEQ ID NO: 64;
f. a heavy chain variable region having the polypeptide sequence of SEQ ID NO: 69, and a light chain variable region having the polypeptide sequence of SEQ ID NO: 70;
g. a heavy chain variable region having the polypeptide sequence of SEQ ID NO: 39, and a light chain variable region having the polypeptide sequence of SEQ ID NO: 40;
h. a heavy chain variable region having the polypeptide sequence of SEQ ID NO: 41, and a light chain variable region having the polypeptide sequence of SEQ ID NO: 42;
i. a heavy chain variable region having the polypeptide sequence of SEQ ID NO: 43, and a light chain variable region having the polypeptide sequence of SEQ ID NO: 44;
j. a heavy chain variable region having the polypeptide sequence of SEQ ID NO: 47, and a light chain variable region having the polypeptide sequence of SEQ ID NO: 48;
k. a heavy chain variable region having the polypeptide sequence of SEQ ID NO: 49, and a light chain variable region having the polypeptide sequence of SEQ ID NO: 50;
l. a heavy chain variable region having the polypeptide sequence of SEQ ID NO: 53, and a light chain variable region having the polypeptide sequence of SEQ ID NO: 54;
m. a heavy chain variable region having the polypeptide sequence of SEQ ID NO: 55, and a light chain variable region having the polypeptide sequence of SEQ ID NO: 56;
n. a heavy chain variable region having the polypeptide sequence of SEQ ID NO: 59, and a light chain variable region having the polypeptide sequence of SEQ ID NO: 60;
o. a heavy chain variable region having the polypeptide sequence of SEQ ID NO: 61, and a light chain variable region having the polypeptide sequence of SEQ ID NO: 62;
p. a heavy chain variable region having the polypeptide sequence of SEQ ID NO: 65, and a light chain variable region having the polypeptide sequence of SEQ ID NO: 66; or
q. a heavy chain variable region having the polypeptide sequence of SEQ ID NO: 67, and a light chain variable region having the polypeptide sequence of SEQ ID NO: 68.

According to another particular aspect, the invention relates to isolated monoclonal antibodies or antigen-binding fragments thereof comprising:
a. a heavy chain having the polypeptide sequence of SEQ ID NO: 37, and a light chain having the polypeptide sequence of SEQ ID NO: 38;
b. a heavy chain having the polypeptide sequence of SEQ ID NO: 11, and a light chain having the polypeptide sequence of SEQ ID NO: 12;
c. a heavy chain having the polypeptide sequence of SEQ ID NO: 17, and a light chain having the polypeptide sequence of SEQ ID NO: 18;
d. a heavy chain having the polypeptide sequence of SEQ ID NO: 23, and a light chain having the polypeptide sequence of SEQ ID NO: 24;
e. a heavy chain having the polypeptide sequence of SEQ ID NO: 29, and a light chain having the polypeptide sequence of SEQ ID NO: 30;
f. a heavy chain having the polypeptide sequence of SEQ ID NO: 35, and a light chain having the polypeptide sequence of SEQ ID NO: 36;
g. a heavy chain having the polypeptide sequence of SEQ ID NO: 5, and a light chain having the polypeptide sequence of SEQ ID NO: 6;
h. a heavy chain having the polypeptide sequence of SEQ ID NO: 7, and a light chain having the polypeptide sequence of SEQ ID NO: 8;
i. a heavy chain having the polypeptide sequence of SEQ ID NO: 9, and a light chain having the polypeptide sequence of SEQ ID NO: 10;
j. a heavy chain having the polypeptide sequence of SEQ ID NO: 13, and a light chain having the polypeptide sequence of SEQ ID NO: 14;
k. a heavy chain having the polypeptide sequence of SEQ ID NO: 15, and a light chain having the polypeptide sequence of SEQ ID NO: 16;
l. a heavy chain having the polypeptide sequence of SEQ ID NO: 19, and a light chain having the polypeptide sequence of SEQ ID NO: 20;
m. a heavy chain having the polypeptide sequence of SEQ ID NO: 21, and a light chain having the polypeptide sequence of SEQ ID NO: 22;
n. a heavy chain having the polypeptide sequence of SEQ ID NO: 25, and a light chain having the polypeptide sequence of SEQ ID NO: 26;
o. a heavy chain having the polypeptide sequence of SEQ ID NO: 27, and a light chain having the polypeptide sequence of SEQ ID NO: 28;
p. a heavy chain having the polypeptide sequence of SEQ ID NO: 31, and a light chain having the polypeptide sequence of SEQ ID NO: 32; or
q. a heavy chain having the polypeptide sequence of SEQ ID NO: 33, and a light chain having the polypeptide sequence of SEQ ID NO: 34.

According to another particular aspect, the invention relates to an isolated humanized antibody or antigen-binding fragment thereof, wherein the antibody or antigen-binding fragment binds to human PHF-tau with a dissociation constant (K_{D}) of 5×10⁻⁹M or less, preferably a K_{D} of 1×10⁻⁹ M or less or 1×10⁻M or less, wherein the K_{D} is measured by surface plasmon resonance analysis, such as by using a Biacore or ProteOn system.

The functional activity of humanized antibodies and antigen-binding fragments thereof that bind PHF-tau can be characterized by methods known in the art and as described herein. Methods for characterizing antibodies and antigen-binding fragments thereof that bind PHF-tau include, but are not limited to, affinity and specificity assays including Biacore, ELISA, and FACS analysis; immunohistochemistry analysis; in vitro cellular assays and in vivo injection assays to determine the efficacy of the antibodies in inhibiting tau seeding; cell cytotoxicity assays to detect the presence of antibody-dependent cell-mediated cytotoxicity (ADCC), and complement dependent cytotoxicity (CDC) activity of the antibodies; etc.

According to particular embodiments, methods for characterizing antibodies and antigen-binding fragments thereof that bind PHF-tau include those described in the Examples below. An exemplary mouse parental antibody of humanized antibodies binding PHF-tau but not control tau is antibody PT3, which has a heavy chain variable region of SEQ ID NO: 1 and a light chain variable region of SEQ ID NO: 2 (see e.g., US Patent No. 9,371,376 which is incorporated by reference in its entirety).

Several well-known methodologies can be employed to determine the binding epitope of the antibodies of the invention. For example, when the structures of both individual components are known, in silico protein-protein docking can be carried out to identify compatible sites of interaction. Hydrogen-deuterium (H/D) exchange can be carried out with the antigen and antibody complex to map regions on the antigen that are bound by the antibody. Segment and point mutagenesis of the antigen can be used to locate amino acids important for antibody binding. The co-crystal structure of an antibody-antigen complex is used to identify residues contributing to the epitope and paratope. According to particular embodiments, methods for determining the binding epitope of antibodies of the invention include those described in Examples below.

Antibodies of the invention can be bispecific or multispecific. An exemplary bispecific antibody can bind two distinct epitopes on PHF-tau or can bind PHF-tau and amyloid beta (Abeta). Another exemplary bispecific antibody can bind PHF-tau and an endogenous blood-brain barrier transcytosis receptor such as insulin receptor, transferring receptor, insulin-like growth factor-1 receptor, and lipoprotein receptor. An exemplary antibody is of IgG1 type.

Immune effector properties of the antibodies of the invention can be enhanced or silenced through Fc modifications by techniques known to those skilled in the art. For example, Fc effector functions such as C1q binding, complement dependent cytotoxicity (CDC), antibody-dependent cell-mediated cytotoxicity (ADCC), phagocytosis, down regulation of cell surface receptors (e.g., B cell receptor; BCR), etc. can be provided and/or controlled by modifying residues in the Fc responsible for these activities. Pharmacokinetic properties can also be enhanced by mutating residues in the Fc domain that extend antibody half-life (Strohl, Curr Opin Biotechnol. 20:685-91, 2009).

Additionally, antibodies of the invention can be post-translationally modified by processes such as glycosylation, isomerization, deglycosylation or non-naturally occurring covalent modification such as the addition of polyethylene glycol moieties and lipidation. Such modifications can occur in vivo or in vitro. For example, the antibodies of the invention can be conjugated to polyethylene glycol (PEGylated) to improve their pharmacokinetic profiles. Conjugation can be carried out by techniques known to those skilled in the art. Conjugation of therapeutic antibodies with PEG has been shown to enhance pharmacodynamics while not interfering with function (Knight et al., Platelets. 15:409-18, 2004; Leong et al., Cytokine. 16:106-19, 2001; Yang et al., Protein Eng. 16:761-70, 2003).

In another general aspect, the invention relates to an isolated polynucleotide encoding a monoclonal antibody or antigen-binding fragment thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding humanized antibodies or antigen-binding fragments thereof of the invention can be altered without changing the amino acid sequences of the proteins. Exemplary isolated polynucleotides are polynucleotides encoding polypeptides comprising the immunoglobulin heavy chain and light chains described in the Examples (e.g., SEQ ID NOs:5-38) and polynucleotides encoding polypeptides comprising the heavy chain variable regions (VH) and light chain variable regions (VL) (e.g., SEQ ID NOs: 39-72). Other polynucleotides which, given the degeneracy of the genetic code or codon preferences in a given expression system, encode the antibodies of the invention are also within the scope of the invention. The isolated nucleic acids of the present invention can be made using well known recombinant or synthetic techniques. DNA encoding the monoclonal antibodies is readily isolated and sequenced using methods known in the art. Where a hybridoma is produced, such cells can serve as a source of such DNA. Alternatively, display techniques wherein the coding sequence and the translation product are linked, such as phage or ribosomal display libraries, can be used.

In another general aspect, the invention relates to a vector comprising an isolated polynucleotide encoding a monoclonal antibody or antigen-binding fragment thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention.

In another general aspect, the invention relates to a host cell comprising an isolated polynucleotide encoding a monoclonal antibody or antigen-binding fragment thereof of the invention. Any host cell known to those skilled in the art in view of the present disclosure can be used for recombinant expression of antibodies or antigen-binding fragments thereof of the invention. Such host cells can be eukaryotic cells, bacterial cells, plant cells or archaeal cells. Exemplary eukaryotic cells can be of mammalian, insect, avian or other animal origins. Mammalian eukaryotic cells include immortalized cell lines such as hybridomas or myeloma cell lines such as SP2/0 (American Type Culture Collection (ATCC), Manassas, Va., CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1SV (Lonza Biologics), CHO-K1 (ATCC CRL-61, Invitrogen) or DG44.

In another general aspect, the invention relates to a method of producing a monoclonal antibody or antigen-binding fragment thereof of the invention, comprising culturing a cell comprising a polynucleotide encoding the monoclonal antibody or antigen-binding fragment thereof under conditions to produce a monoclonal antibody or antigen-binding fragment thereof of the invention, and recovering the antibody or antigen-binding fragment thereof from the cell or cell culture (e.g., from the supernatant). Expressed antibodies or antigen-binding fragments thereof can be harvested from the cells and purified according to conventional techniques known in the art.

### Pharmaceutical Compositions and Methods of Treatment

Anti-PHF-tau antibodies of the invention or fragments thereof of the invention can be used to treat, reduce or prevent symptoms in patients having a neurodegenerative disease that involves pathological aggregation of tau within the brain, or a tauopathy, such as patients suffering from AD.

Thus, in another general aspect, the invention relates to a pharmaceutical composition comprising an isolated monoclonal antibody or antigen-binding fragment thereof of the invention and a pharmaceutically acceptable carrier.

In another general aspect, the invention relates to a method of treating or reducing symptoms of a disease, disorder or condition, such as a tauopathy, in a subject in need thereof, comprising administering to the subject a pharmaceutical composition of the invention.

In another general aspect, the invention relates to a method of reducing pathological tau aggregation or spreading of tauopathy in a subject in need thereof, comprising administering to the subject a pharmaceutical composition of the invention.

According to embodiments of the invention, the pharmaceutical composition comprises a therapeutically effective amount of the monoclonal anti-PHF-tau antibody or antigen-binding fragment thereof. As used herein with reference to humanized anti-PHF-tau antibodies or antigen-binding fragments thereof, a therapeutically effective amount means an amount of the monoclonal anti-PHF-tau antibody or antigen-binding fragment thereof that results in treatment of a disease, disorder, or condition; prevents or slows the progression of the disease, disorder, or condition; or reduces or completely alleviates symptoms associated with the immune disease, disorder, or condition.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

According to particular embodiments, the disease, disorder or condition to be treated is a tauopathy. According to more particular embodiments, the disease, disorder or condition to be treated, includes, but is not limited to, familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, or dementia pugulistica (boxing disease).

A tauopathy-related behavioral phenotype includes, but is not limited to, cognitive impairments, early personality change and disinhibition, apathy, abulia, mutism, apraxia, perseveration, stereotyped movements/behaviors, hyperorality, disorganization, inability to plan or organize sequential tasks, selfishness/callousness, antisocial traits, a lack of empathy, halting, agrammatic speech with frequent paraphasic errors but relatively preserved comprehension, impaired comprehension and word-finding deficits, slowly progressive gait instability, retropulsions, freezing, frequent falls, non-levodopa responsive axial rigidity, supranuclear gaze palsy, square wave jerks, slow vertical saccades, pseudobulbar palsy, limb apraxia, dystonia, cortical sensory loss, and tremor.

Patients amenable to treatment include, but are not limited to, asymptomatic individuals at risk of AD or other tauopathy, as well as patients presently showing symptoms. Patients amenable to treatment include individuals who have a known genetic risk of AD, such as a family history of AD or presence of genetic risk factors in the genome. Exemplary risk factors are mutations in the amyloid precursor protein (APP), especially at position 717 and positions 670 and 671 (Hardy and Swedish mutations, respectively). Other risk factors are mutations in the presenilin genes PS1 and PS2 and in ApoE4, family history of hypercholesterolemia or atherosclerosis. Individuals presently suffering from AD can be recognized from characteristic dementia by the presence of risk factors described above. In addition, a number of diagnostic tests are available to identify individuals who have AD. These include measurement of cerebrospinal fluid tau and Abeta 42 levels. Elevated tau and decreased Abeta 42 levels signify the presence of AD. Individuals suffering from AD can also be diagnosed by AD and Related Disorders Association criteria.

Anti-PHF-tau antibodies of the invention are suitable both as therapeutic and prophylactic agents for treating or preventing neurodegenerative diseases that involve pathological aggregation of tau, such as AD or other tauopathies. In asymptomatic patients, treatment can begin at any age (e.g., at about 10, 15, 20, 25, 30 years). Usually, however, it is not necessary to begin treatment until a patient reaches about 40, 50, 60, or 70 years. Treatment typically entails multiple dosages over a period of time. Treatment can be monitored by assaying antibody or activated T-cell or B-cell responses to the therapeutic agent over time. If the response falls, a booster dosage can be indicated.

In prophylactic applications, pharmaceutical compositions or medicaments are administered to a patient susceptible to, or otherwise at risk of, AD in an amount sufficient to eliminate or reduce the risk, lessen the severity, or delay the outset of the disease, including biochemical, histologic and/or behavioral symptoms of the disease, its complications and intermediate pathological phenotypes presented during development of the disease. In therapeutic applications, compositions or medicaments are administered to a patient suspected of, or already suffering from, such a disease in an amount sufficient to reduce, arrest, or delay any of the symptoms of the disease (biochemical, histologic and/or behavioral). Administration of a therapeutic can reduce or eliminate mild cognitive impairment in patients that have not yet developed characteristic Alzheimer's pathology.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

The antibodies of the invention can be prepared as pharmaceutical compositions containing a therapeutically effective amount of the antibody as an active ingredient in a pharmaceutically acceptable carrier. The carrier can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They can be sterilized by conventional, well-known sterilization techniques (e.g., filtration). The compositions can contain pharmaceutically acceptable auxiliary substances as required to approximate physiological conditions such as pH adjusting and buffering agents, stabilizing, thickening, lubricating and coloring agents, etc. The concentration of the antibodies of the invention in such pharmaceutical formulation can vary widely, i.e., from less than about 0.5%, usually at or at least about 1% to as much as 15 or 20% by weight and will be selected primarily based on required dose, fluid volumes, viscosities, etc., according to the particular mode of administration selected.

The mode of administration for therapeutic use of the antibodies of the invention can be any suitable route that delivers the agent to the host. For example, the compositions described herein can be formulated to be suitable for parenteral administration, e.g., intradermal, intramuscular, intraperitoneal, intravenous, subcutaneous, intranasal or intracranial administration, or they can be administered into the cerebrospinal fluid of the brain or spine.

The treatment can be given in a single dose schedule, or as a multiple dose schedule in which a primary course of treatment can be with 1-10 separate doses, followed by other doses given at subsequent time intervals required to maintain and or reinforce the response, for example, at 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms or reduce severity of disease.

The antibodies of the invention can be lyophilized for storage and reconstituted in a suitable carrier prior to use. This technique has been shown to be effective with antibody and other protein preparations and art-known lyophilization and reconstitution techniques can be employed.

According to particular embodiments, a composition used in the treatment of a tauopathy can be used in combination with other agents that are effective for treatment of related neurodegenerative diseases. In the case of AD, antibodies of the invention can be administered in combination with agents that reduce or prevent the deposition of amyloid-beta (Abeta). It is possible that PHF-tau and Abeta pathologies are synergistic. Therefore, combination therapy targeting the clearance of both PHF-tau and Abeta and Abeta-related pathologies at the same time can be more effective than targeting each individually. In the case of Parkinson's Disease and related neurodegenerative diseases, immune modulation to clear aggregated forms of the alpha-synuclein protein is also an emerging therapy. A combination therapy which targets the clearance of both tau and alpha-synuclein proteins simultaneously can be more effective than targeting either protein individually.

In another general aspect, the invention relates to a method of producing a pharmaceutical composition comprising a monoclonal antibody or antigen-binding fragment thereof of the invention, comprising combining a monoclonal antibody or antigen-binding fragment thereof with a pharmaceutically acceptable carrier to obtain the pharmaceutical composition.

### Diagnostic Methods and Kits

Monoclonal anti-PHF-tau antibodies of the invention can be used in methods of diagnosing AD or other tauopathies in a subject.

Thus, in another general aspect, the invention relates to methods of detecting the presence of PHF-tau in a subject and methods of diagnosing tauopathies in a subject by detecting the presence of PHF-tau in the subject using a monoclonal antibody or antigen-binding fragment thereof of the invention.

Phosphorylated tau can be detected in a biological sample from a subject (e.g., blood, serum, plasma, interstitial fluid, or cerebral spinal fluid sample) by contacting the biological sample with the diagnostic antibody reagent and detecting binding of the diagnostic antibody reagent to phosphorylated tau in the sample from the subject. Assays for carrying out the detection include well-known methods such as ELISA, immunohistochemistry, western blot, or in vivo imaging.

Diagnostic antibodies or similar reagents can be administered by intravenous injection into the body of the patient, or directly into the brain by any suitable route that delivers the agent to the host. The dosage of antibody should be within the same ranges as for treatment methods. Typically, the antibody is labeled, although in some methods, the primary antibody with affinity for phosphorylated tau is unlabeled, and a secondary labeling agent is used to bind to the primary antibody. The choice of label depends on the means of detection. For example, a fluorescent label is suitable for optical detection. Use of paramagnetic labels is suitable for tomographic detection without surgical intervention. Radioactive labels can also be detected using PET or SPECT.

Diagnosis is performed by comparing the number, size, and/or intensity of labeled PHF-tau, tau aggregates, and/or neurofibrillary tangles in a sample from the subject or in the subject, to corresponding baseline values. The baseline values can represent the mean levels in a population of healthy individuals. Baseline values can also represent previous levels determined in the same subject.

The diagnostic methods described above can also be used to monitor a subject's response to therapy by detecting the presence of phosphorylated tau in a subject before, during or after the treatment. A decrease in values relative to baseline signals a positive response to treatment. Values can also increase temporarily in biological fluids as pathological tau is being cleared from the brain.

The present invention is further directed to a kit for performing the above described diagnostic and monitoring methods. Typically, such kits contain a diagnostic reagent such as the antibodies of the invention, and optionally a detectable label. The diagnostic antibody itself can contain the detectable label (e.g., fluorescent molecule, biotin, etc.) which is directly detectable or detectable via a secondary reaction (e.g., reaction with streptavidin).

Alternatively, a second reagent containing the detectable label cab be used, where the second reagent has binding specificity for the primary antibody. In a diagnostic kit suitable for measuring PHF-tau in a biological sample, the antibodies of the kit can be supplied pre-bound to a solid phase, such as to the wells of a microtiter dish.

The contents of all cited references (including literature references, issued patents, published patent applications, and co-pending patent applications) cited throughout this application are hereby expressly incorporated by reference.

### EMBODIMENTS

The invention provides also the following non-limiting embodiments.

Embodiment 1 is an isolated monoclonal antibody or antigen-binding fragment thereof comprising a heavy chain variable region having a polypeptide sequence selected from SEQ ID NO: 71, 45, 51, 57, 63, 69, 39, 41, 43, 47, 49, 53, 55, 59, 61, 65, or 67, or a light chain variable region having a polypeptide sequence selected from SEQ ID NO: 72, 46, 52, 58, 64, 70, 40, 42, 44, 48, 50, 54, 56, 62, 66, or 68, wherein the monoclonal antibody or antigen-binding fragment thereof specifically binds paired helical filament (PHF)-tau, preferably human PHF-tau.

Embodiment 2 is the isolated monoclonal antibody or antigen-binding fragment thereof of embodiment 1, wherein the monoclonal antibody or antigen-binding fragment thereof comprises:
a. a heavy chain variable region having the polypeptide sequence of SEQ ID NO: 71, and a light chain variable region having the polypeptide sequence of SEQ ID NO: 72;
b. a heavy chain variable region having the polypeptide sequence of SEQ ID NO:45, and a light chain variable region having the polypeptide sequence of SEQ ID NO:46;
c. a heavy chain variable region having the polypeptide sequence of SEQ ID NO: 51, and a light chain variable region having the polypeptide sequence of SEQ ID NO: 52;
d. a heavy chain variable region having the polypeptide sequence of SEQ ID NO: 57, and a light chain variable region having the polypeptide sequence of SEQ ID NO: 58;
e. a heavy chain variable region having the polypeptide sequence of SEQ ID NO: 63, and a light chain variable region having the polypeptide sequence of SEQ ID NO: 64;
f. a heavy chain variable region having the polypeptide sequence of SEQ ID NO: 69, and a light chain variable region having the polypeptide sequence of SEQ ID NO: 70;
g. a heavy chain variable region having the polypeptide sequence of SEQ ID NO: 39, and a light chain variable region having the polypeptide sequence of SEQ ID NO: 40;
h. a heavy chain variable region having the polypeptide sequence of SEQ ID NO: 41, and a light chain variable region having the polypeptide sequence of SEQ ID NO: 42;
i. a heavy chain variable region having the polypeptide sequence of SEQ ID NO: 43, and a light chain variable region having the polypeptide sequence of SEQ ID NO: 44;
j. a heavy chain variable region having the polypeptide sequence of SEQ ID NO: 47, and a light chain variable region having the polypeptide sequence of SEQ ID NO: 48;
k. a heavy chain variable region having the polypeptide sequence of SEQ ID NO: 49, and a light chain variable region having the polypeptide sequence of SEQ ID NO: 50;
l. a heavy chain variable region having the polypeptide sequence of SEQ ID NO: 53, and a light chain variable region having the polypeptide sequence of SEQ ID NO: 54;
m. a heavy chain variable region having the polypeptide sequence of SEQ ID NO: 55, and a light chain variable region having the polypeptide sequence of SEQ ID NO: 56;
n. a heavy chain variable region having the polypeptide sequence of SEQ ID NO: 59, and a light chain variable region having the polypeptide sequence of SEQ ID NO: 60;
o. a heavy chain variable region having the polypeptide sequence of SEQ ID NO: 61, and a light chain variable region having the polypeptide sequence of SEQ ID NO: 62;
p. a heavy chain variable region having the polypeptide sequence of SEQ ID NO: 65, and a light chain variable region having the polypeptide sequence of SEQ ID NO: 66; or
q. a heavy chain variable region having the polypeptide sequence of SEQ ID NO: 67, and a light chain variable region having the polypeptide sequence of SEQ ID NO: 68.

Embodiment 3 is the isolated monoclonal antibody or antigen-binding fragment thereof of embodiment 1 or 2, wherein the monoclonal antibody or antigen-binding fragment thereof comprises:
a. a heavy chain having the polypeptide sequence of SEQ ID NO: 37, and a light chain having the polypeptide sequence of SEQ ID NO: 38;
b. a heavy chain having the polypeptide sequence of SEQ ID NO: 11, and a light chain having the polypeptide sequence of SEQ ID NO: 12;
c. a heavy chain having the polypeptide sequence of SEQ ID NO: 17, and a light chain having the polypeptide sequence of SEQ ID NO: 18;
d. a heavy chain having the polypeptide sequence of SEQ ID NO: 23, and a light chain having the polypeptide sequence of SEQ ID NO: 24;
e. a heavy chain having the polypeptide sequence of SEQ ID NO: 29, and a light chain having the polypeptide sequence of SEQ ID NO: 30;
f. a heavy chain having the polypeptide sequence of SEQ ID NO: 35, and a light chain having the polypeptide sequence of SEQ ID NO: 36;
g. a heavy chain having the polypeptide sequence of SEQ ID NO: 5, and a light chain having the polypeptide sequence of SEQ ID NO: 6;
h. a heavy chain having the polypeptide sequence of SEQ ID NO: 7, and a light chain having the polypeptide sequence of SEQ ID NO: 8;
i. a heavy chain having the polypeptide sequence of SEQ ID NO: 9, and a light chain having the polypeptide sequence of SEQ ID NO: 10;
j. a heavy chain having the polypeptide sequence of SEQ ID NO: 13, and a light chain having the polypeptide sequence of SEQ ID NO: 14;
k. a heavy chain having the polypeptide sequence of SEQ ID NO: 15, and a light chain having the polypeptide sequence of SEQ ID NO: 16;
l. a heavy chain having the polypeptide sequence of SEQ ID NO: 19, and a light chain having the polypeptide sequence of SEQ ID NO: 20;
m. a heavy chain having the polypeptide sequence of SEQ ID NO: 21, and a light chain having the polypeptide sequence of SEQ ID NO: 22;
n. a heavy chain having the polypeptide sequence of SEQ ID NO: 25, and a light chain having the polypeptide sequence of SEQ ID NO: 26;
o. a heavy chain having the polypeptide sequence of SEQ ID NO: 27, and a light chain having the polypeptide sequence of SEQ ID NO: 28;
p. a heavy chain having the polypeptide sequence of SEQ ID NO: 31, and a light chain having the polypeptide sequence of SEQ ID NO: 32; or
q. a heavy chain having the polypeptide sequence of SEQ ID NO: 33, and a light chain having the polypeptide sequence of SEQ ID NO: 34.

Embodiment 4 is an isolated nucleic acid encoding the monoclonal antibody or antigen-binding fragment thereof of any one of embodiments 1 to 3.

Embodiment 5 is a vector comprising the isolated nucleic acid of embodiment 4.

Embodiment 6 is a host cell comprising the nucleic acid of embodiment 5.

Embodiment 7 is a pharmaceutical composition comprising the isolated monoclonal antibody or antigen-binding fragment thereof of any one of embodiments 1 to 3 and a pharmaceutically acceptable carrier.

Embodiment 8 is a method of reducing pathological tau aggregation or spreading of tauopathy in a subject in need thereof, comprising administering to the subject the pharmaceutical composition of embodiment 7.

Embodiment 9 is a method of treating a tauopathy, in a subject in need thereof, comprising administering to the subject the pharmaceutical composition of embodiment 7.

Embodiment 10 is the method of embodiment 9, further comprising administering to the subject an additional agent for treating the tauopathy in the subject in need thereof.

Embodiment 11 is a method of treating a tauopathy in a subject in need thereof, comprising administering to the subject the pharmaceutical composition of embodiment 7, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

Embodiment 12 is the method of embodiment 11, further comprising administering to the subject an additional agent for treating the tauopathy in the subject in need thereof.

Embodiment 13 is a method of producing the monoclonal antibody or antigen-binding fragment thereof of any one of embodiments 1 to 3, comprising culturing a cell comprising a nucleic acid encoding the monoclonal antibody or antigen-binding fragment thereof under conditions to produce the monoclonal antibody or antigen-binding fragment thereof and recovering the monoclonal antibody or antigen-binding fragment thereof from the cell or cell culture.

Embodiment 14 is a method of producing a pharmaceutical composition comprising the monoclonal antibody or antigen-binding fragment thereof of any one of embodiments 1 to 3, comprising combining the monoclonal antibody or antigen-binding fragment thereof with a pharmaceutically acceptable carrier to obtain the pharmaceutical composition.

Embodiment 15 is an isolated monoclonal antibody or antigen-binding fragment thereof of any one of embodiments 1 to 3 for use in treating a tauopathy, in a subject in need thereof.

Embodiment 16 is an isolated monoclonal antibody or antigen-binding fragment thereof of any one of embodiments 1 to 3 or the pharmaceutical composition of embodiment 7 for use in treating a tauopathy, such as familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, or dementia pugulistica (boxing disease), in a subject in need thereof.

Embodiment 17 is a use of an isolated monoclonal antibody or antigen-binding fragment thereof of any one of embodiments 1 to 3 for manufacturing a medicament in treating a tauopathy in a subject in need thereof.

Embodiment 18 is a use of an isolated monoclonal antibody or antigen-binding fragment thereof of any one of embodiments 1 to 3 for manufacturing a medicament for treating a tauopathy, such as familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Sträussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, or dementia pugulistica (boxing disease), in a subject in need thereof.

Embodiment 19 is a method of detecting the presence of PHF-tau in a biological sample from a subject, comprising contacting the biological sample with the isolated monoclonal antibody or antigen-binding fragment thereof of any one of embodiments 1 to 3, and detecting binding of the monoclonal antibody or antigen-binding fragment thereof to PHF-tau in the sample from the subject.

Embodiment 20 is the method of embodiment 19, wherein the biological sample is a blood, serum, plasma, interstitial fluid, or cerebral spinal fluid sample.

Embodiment 21 is a method of diagnosing a tauopathy in a subject by detecting the presence of PHF-tau in a biological sample from the subject, comprising contacting the biological sample with the isolated monoclonal antibody or antigen-binding fragment thereof of any one of embodiments 1 to 3, and detecting binding of the antibody or antigen-binding fragment to PHF-tau in the sample from the subject.

### EXAMPLES

The following examples of the invention are to further illustrate the nature of the invention. It should be understood that the following examples do not limit the invention and that the scope of the invention is to be determined by the appended claims.

### Example 1

### Outline of the Humanization Process and Evaluation of Binding Properties

### Humanization of Anti-Tau Antibody PT3

The parental antibody PT3 was humanized (see, e.g., WO2013/3096380; U.S. Pat. No. 10,000,559B2). To find the best combination of humanized heavy and light chains, the most aligned human germline heavy chain and human germline light chain frame works were selected for the antibody humanization. Human J segment for VL was chosen by comparing the parental J segment sequence to human J segment sequences to maximize sequence identity. The human J segment for VH included a G105V mutation.

Selection of Humanized Variants by SPR on E.coli-Produced Fabs.

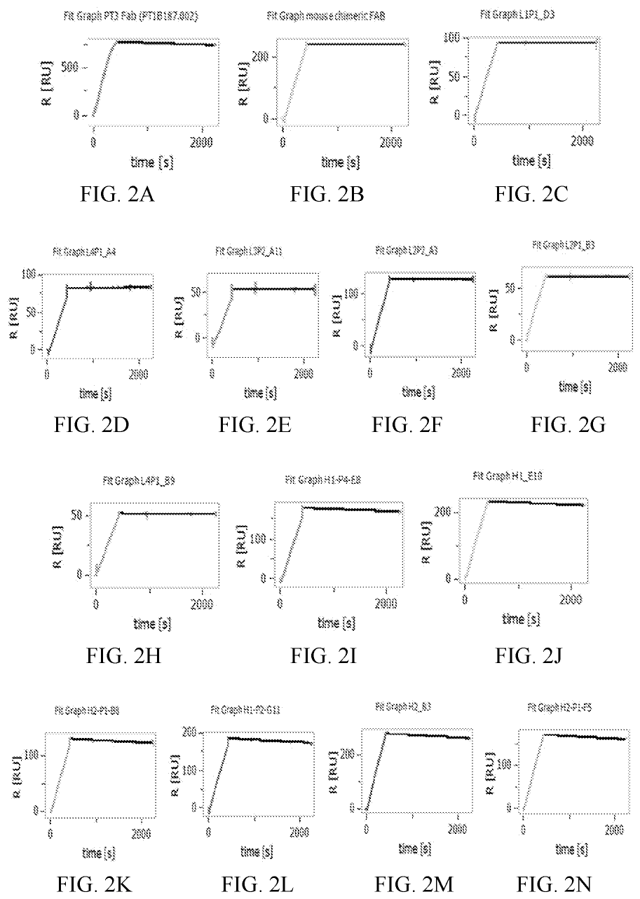

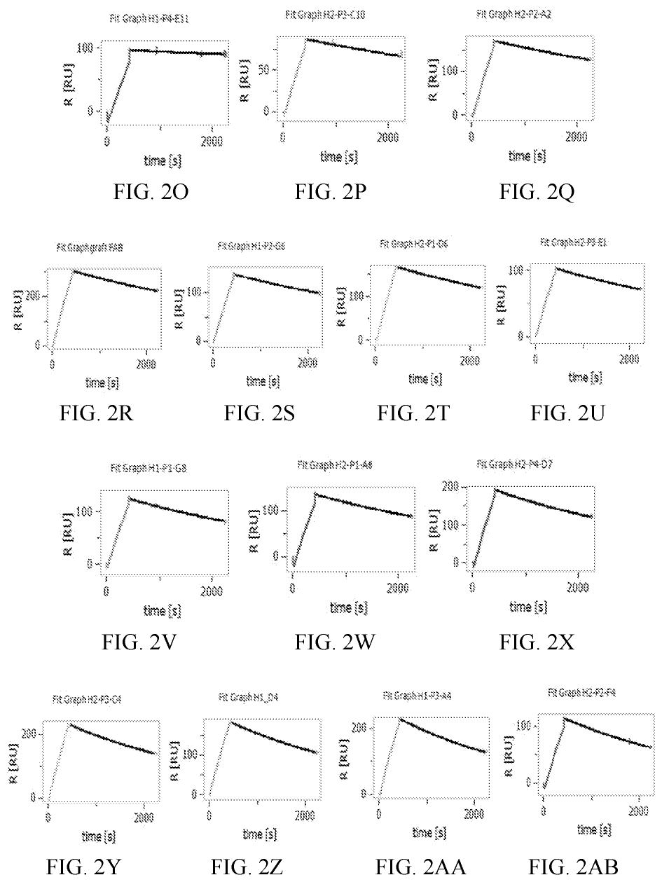

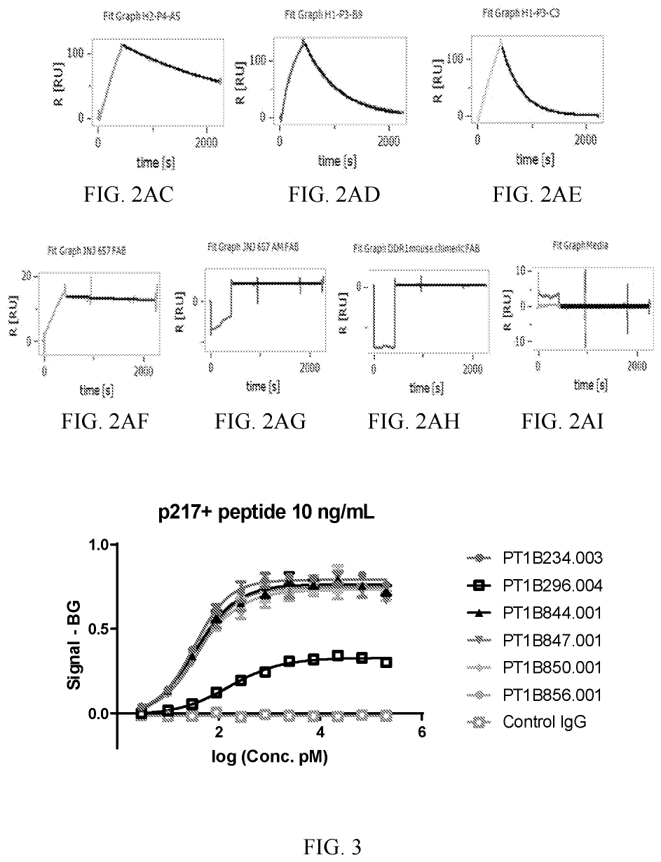

Method: SPR based off-rate analysis experiments of the humanized Fab supernatants produced from E. coli were performed using MASS-2 instrument. Briefly, the high capacity amine (HCA) sensor chip was activated using EDC/NHS mixture and the surface was coated with neutravidin (>4000 RU) using amine-coupling chemistry and the surface was deactivated using Ethanolamine. Following this, the biotinylated phospho-tau peptide (NPT-6) was captured through neutravidin at different levels (40-100 RU). To measure the binding of the Fabs to the captured phospho-tau peptide, the crude Fab supernatants were injected as neat solutions over the peptide surface and the association/dissociation profiles were monitored. Following dissociation, the surface was regenerated with phosphoric acid for next round of Fab interactions. The sensorgrams for the binding of Fabs to phospho-peptide were analyzed using off-rate analysis method to determine the rank order based on off-rate values. Several Fab supernatants retained similar or better off-rates compared to the parent mouse antibody and these were selected for IgG conversion. The binding sensorgrams of the Fab panel are shown in FIGS. 2A-2AI and the off-rates are provided in Table 1.

TABLE 1
MASS-2 SPR off-rate results for E. coli
Fab supernatants binding to NPT-6 peptide

| Fab Supernatants | Kd [1/s]** |
|---|---|
| PTS purified Fab (PT1B187.002) | 2.73E−05 |
| mouse chimeric FAB | <2.85E−05 |
| L1-P1-D3 | <2.85E−05 |
| L4-P1-A4 | <2.85E−05 |
| L3-P2-A11 | <2.85E−05 |
| L2-P2-A3 | <2.85E−05 |
| L2-P1-B3 | <2.85E−05 |
| L4-P1-B9 | <2.85E−05 |
| H1-P4-E8 | <2.85E−05 |
| H1-E10 | 3.11E−05 |
| H2-P1-B8 | 3.37E−05 |
| H1-P2-G11 | 3.41E−05 |
| H2-B3 | 3.49E−05 |
| H2-P1-F5 | 3.50E−05 |
| H1-P4-E11 | 4.05E−05 |
| H2-P3-C10 | 1.50E−04 |
| H2-P2-A2 | 1.65E−04 |
| graft FAB | 1.76E−04 |
| H1-P2-G6 | 1.83E−04 |
| H2-P1-D6 | 1.89E−04 |
| H2-P3-E1 | 2.08E−04 |
| H1-P1-G8 | 2.36E−04 |
| H2-P1-A8 | 2.40E−04 |
| H2-P4-D7 | 2.60E−04 |
| H2-P3-C4 | 2.82E−04 |
| H1-D4 | 3.02E−04 |
| H1-P3-A4 | 3.27E−04 |
| H2-P2-F4 | 3.28E−04 |
| H2-P4-A5 | 3.90E−04 |
| H1-P3-B9 | 1.59E−03 |
| H1-P3-C3 | 2.83E−03 |

Back mutation libraries were created through molecular biology and library clones were tested for binding to the bt-peptide through ELISA with signals compared against the fully murine parent molecule. Signals exhibiting binding greater than 80% of the murine parent were selected for sequencing. Sequences were analyzed. 21 human adapted heavy chains and 5 human adapted light chains were selected. Further screening, including full ELISA binding curves and SPR off rate analysis of the selected clones was performed (FIGS. 2A-2AI). 5 heavy chains and 3 light chains were chosen based on these results. The full matrix of heavy chain and light chain were also tested as Fabs in ELISA and confirmed the previous results and conclusion. These final 5 heavy and 3 light chains were combined to make 15 matrixed clones (Tables 2 and 3) and expressed as monoclonal IgG1 molecules and further tested as purified mAbs for antigen binding along with various developmental assays (Tm/Tagg, CIC, SEC, AC-SINS, Affinity & In-Silico EpiVax scores). Based on this data, an antibody PT1B844 demonstrated comparable or improved bt-peptide binding and biophysical properties to the parental mouse antibody and was chosen for further development. An IgG1 YTE version PT1B916 was also created (Tables 2 and 3).

Both PT1B844 and PT1B916 contained a potentially unwanted (non-human) J-segment point mutation of G105V. To address this potential issue, mAbs were created to convert the position back to human. The new clones were expressed as both IgG1 and IgG1 YTEs for further characterization.

TABLE 2
Heavy chain (HC) and light chain (LC) sequences
of humanized monoclonal antibodies

| | | SEQ |
|---|---|---|
| mAb | | ID |
| component | Sequence | NO: |
| PT3 HC | EVKLVESGGDLVKPGGSLKLSCAASGFTFSSYAMSWVRQNPEKRLEWVAS | 1 |
| | ISKGGNTYYPNSVKGRFTISRDNARNILYLQMSSLRSEDTALYYCARGWG | |
| | DYGWFAYWGQVTLVTVSA | |
| PT3 LC | DIKMTQSPSSMYASLGERVTITCKASQDINRYLNWFQQKPGKSPKTLIYR | 2 |
| | ANRLLDGVPSRFSGSGSGQDYSLTISSLDYEDMGIYYCLQYDEFPLTFGD | |
| | GTKLELK | |
| Graft HC | EVQLVESGGGLVKPGGSLRLSCAASGFTFSSYAMSWVRQAPGKGLEWVSS | 3 |
| | ISKGGNTYYADSVKGRFTISRDNAKNSLYLQMNSLRAEDTAVYYCARGWG | |
| | DYGWFAYWGQVTLVTVSS | |
| Graft LC | DIQMTQSPSSLSASVGDRVTITCKASQDINRYLNWFQQKPGKAPKSLIYR | 4 |
| | ANRLLDGVPSRFSGSGSGTDFTLTISSLQPEDFATYYCLQYDEFPLTFGQ | |
| | GTKLEIK | |
| PT1B841 | EVQLVESGGGLVKPGGSLRLSCAASGFTFSSYAMSWVRQAPGKGLEWVSS | 5 |
| HC | ISKGGNTYYADSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B841 | DIQMTQSPSSLSASVGDRVTITCKASQDINRYLNWFQQKPGKAPKSLIYR | 6 |
| LC | ANRLLDGVPSRFSGSGSGTDFTLTISSLQPEDFATYYCLQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B842 | EVQLVESGGGLVKPGGSLRLSCAASGFTFSSYAMSWVRQAPGKGLEWVAS | 7 |
| HC | ISKGGNTYYPDSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B842 | DIQMTQSPSSLSASVGDRVTITCKASQDINRYLNWFQQKPGKAPKSLIYR | 8 |
| LC | ANRLLSGVPSRFSGSGSGTDFTLTISSLQPEDMATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B843 | EVQLVESGGGLVKPGGSLRLSCAASGFTFSSYAMSWVRQAPGKGLEWVAS | 9 |
| HC | ISKGGNTYYPDSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B843 | DIQMTQSPSSLSASVGDRVTITCKASQDINRYLNWFQQKPGKSPKTLIYR | 10 |
| LC | ANRLLSGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B844 | EVQLVESGGGLVKPGGSLRLSCAASGFTFSSYAMSWVRQAPGKGLEWVAS | 11 |
| HC | ISKGGNTYYPDSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B844 | DIQMTQSPSSLSASVGDRVTITCKASQDINRYLNWFQQKPGKAPKTLIYR | 12 |
| LC | ANRLLDGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B845 | EVQLVESGGGLVKPGGSLRLSCAASGFTFSSYAMSWVRQAPGKRLEWVAS | 13 |
| HC | ISKGGNTYYPNSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B845 | DIQMTQSPSSLSASVGDRVTITCKASQDINRYLNWFQQKPGKAPKSLIYR | 14 |
| LC | ANRLLSGVPSRFSGSGSGTDFTLTISSLQPEDMATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B846 | EVQLVESGGGLVKPGGSLRLSCAASGFTFSSYAMSWVRQAPGKRLEWVAS | 15 |
| HC | ISKGGNTYYPNSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B846 | DIQMTQSPSSLSASVGDRVTITCKASQDINRYLNWFQQKPGKSPKTLIYR | 16 |
| LC | ANRLLSGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B847 | EVQLVESGGGLVKPGGSLRLSCAASGFTFSSYAMSWVRQAPGKRLEWVAS | 17 |
| HC | ISKGGNTYYPNSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B847 | DIQMTQSPSSLSASVGDRVTITCKASQDINRYLNWFQQKPGKAPKTLIYR | 18 |
| LC | ANRLLDGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B848 | EVQLVESGGGLVKPGGSLRLSCAASGFTFSSYAMSWVRQAPGKRLEWVAS | 19 |
| HC | ISKGGNTYYPDSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B848 | DIQMTQSPSSLSASVGDRVTITCKASQDINRYLNWFQQKPGKAPKSLIYR | 20 |
| LC | ANRLLSGVPSRFSGSGSGTDFTLTISSLQPEDMATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B849 | EVQLVESGGGLVKPGGSLRLSCAASGFTFSSYAMSWVRQAPGKRLEWVAS | 21 |
| HC | ISKGGNTYYPDSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B849 | DIQMTQSPSSLSASVGDRVTITCKASQDINRYLNWFQQKPGKSPKTLIYR | 22 |
| LC | ANRLLSGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B850 | EVQLVESGGGLVKPGGSLRLSCAASGFTFSSYAMSWVRQAPGKRLEWVAS | 23 |
| HC | ISKGGNTYYPDSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B850 | DIQMTQSPSSLSASVGDRVTITCKASQDINRYLNWFQQKPGKAPKTLIYR | 24 |
| LC | ANRLLDGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B851 | EVQLVESGGGLVKPGGSLRLSCAASGFTFSSYAMSWVRQAPGKGLEWVAS | 25 |
| HC | ISKGGNTYYPNSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B851 | DIQMTQSPSSLSASVGDRVTITCKASQDINRYLNWFQQKPGKAPKSLIYR | 26 |
| LC | ANRLLSGVPSRFSGSGSGTDFTLTISSLQPEDMATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B852 | EVQLVESGGGLVKPGGSLRLSCAASGFTFSSYAMSWVRQAPGKGLEWVAS | 27 |
| HC | ISKGGNTYYPNSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B852 | DIQMTQSPSSLSASVGDRVTITCKASQDINRYLNWFQQKPGKSPKTLIYR | 28 |
| LC | ANRLLSGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B853 | EVQLVESGGGLVKPGGSLRLSCAASGFTFSSYAMSWVRQAPGKGLEWVAS | 29 |
| HC | ISKGGNTYYPNSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B853 | DIQMTQSPSSLSASVGDRVTITCKASQDINRYLNWFQQKPGKAPKTLIYR | 30 |
| LC | ANRLLDGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B854 | EVQLVESGGGLVKPGGSLRLSCAASGFTFSSYAMSWVRQNPGKGLEWVAS | 31 |
| HC | ISKGGNTYYPDSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B854 | DIQMTQSPSSLSASVGDRVTITCKASQDINRYLNWFQQKPGKAPKSLIYR | 32 |
| LC | ANRLLSGVPSRFSGSGSGTDFTLTISSLQPEDMATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B855 | EVQLVESGGGLVKPGGSLRLSCAASGFTFSSYAMSWVRQNPGKGLEWVAS | 33 |
| HC | ISKGGNTYYPDSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B855 | DIQMTQSPSSLSASVGDRVTITCKASQDINRYLNWFQQKPGKSPKTLIYR | 34 |
| LC | ANRLLSGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B856 | EVQLVESGGGLVKPGGSLRLSCAASGFTFSSYAMSWVRQNPGKGLEWVAS | 35 |
| HC | ISKGGNTYYPDSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B856 | DIQMTQSPSSLSASVGDRVTITCKASQDINRYLNWFQQKPGKAPKTLIYR | 36 |
| LC | ANRLLDGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PT1B916 | EVQLVESGGGLVKPGGSLRLSCAASGFTFSSYAMSWVRQAPGKGLEWVAS | 37 |
| HC | ISKGGNTYYPDSVKGRFTISRDNAKNSLYLQMNSLRAEDTAVYYCARGWG | |
| | DYGWFAYWGQVTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYI | |
| | CNVNHKPSNTKVDKKVEPKSCDKTHTCPPCPAPELLGGPSVFLFPPKPKD | |
| | TLYITREPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVY | |
| | TLPPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| PT1B916 | DIQMTQSPSSLSASVGDRVTITCKASQDINRYLNWFQQKPGKAPKTLIYR | 38 |
| LC | ANRLLDGVPSRFSGSGSGTDFTLTISSLQPEDFATYYCQQYDEFPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |

TABLE 3
Heavy Chain and Light Chain Variable Regions (VH and VL,
respectively) of humanized monoclonal antibodies

| | | SEQ |
|---|---|---|
| mAb | | ID |
| component | Sequence | NO: |
| PT1B841 | EVQLVESGGGLVKPGGSLRLSCAASGFTFSSYAMSWVRQAPGKGLEWVSSI | 39 |
| VH | SKGGNTYYADSVKGRFTISRDNAKNSLYLQMNSLRAEDTAVYYCARGWGDY | |
| | GWFAYWGQVTLVTVSS | |
| PT1B841 | DIQMTQSPSSLSASVGDRVTITCKASQDINRYLNWFQQKPGKAPKSLIYRA | 40 |
| VL | NRLLDGVPSRFSGSGSGTDFTLTISSLQPEDFATYYCLQYDEFPLTFGQGT | |
| | KLEIK | |
| PT1B842 | EVQLVESGGGLVKPGGSLRLSCAASGFTFSSYAMSWVRQAPGKGLEWVASI | 41 |
| VH | SKGGNTYYPDSVKGRFTISRDNAKNSLYLQMNSLRAEDTAVYYCARGWGDY | |
| | GWFAYWGQVTLVTVSS | |
| PT1B842 | DIQMTQSPSSLSASVGDRVTITCKASQDINRYLNWFQQKPGKAPKSLIYRA | 42 |
| VL | NRLLSGVPSRFSGSGSGTDFTLTISSLQPEDMATYYCQQYDEFPLTFGQGT | |
| | KLEIK | |
| PT1B843 | EVQLVESGGGLVKPGGSLRLSCAASGFTFSSYAMSWVRQAPGKGLEWVASI | 43 |
| VH | SKGGNTYYPDSVKGRFTISRDNAKNSLYLQMNSLRAEDTAVYYCARGWGDY | |
| | GWFAYWGQVTLVTVSS | |
| PT1B843 | DIQMTQSPSSLSASVGDRVTITCKASQDINRYLNWFQQKPGKSPKTLIYRA | 44 |
| VL | NRLLSGVPSRFSGSGSGTDFTLTISSLQPEDFATYYCQQYDEFPLTFGQGT | |
| | KLEIK | |
| PT1B844 | EVQLVESGGGLVKPGGSLRLSCAASGFTFSSYAMSWVRQAPGKGLEWVASI | 45 |
| VH | SKGGNTYYPDSVKGRFTISRDNAKNSLYLQMNSLRAEDTAVYYCARGWGDY | |
| | GWFAYWGQVTLVTVSS | |
| PT1B844 | DIQMTQSPSSLSASVGDRVTITCKASQDINRYLNWFQQKPGKAPKTLIYRA | 46 |
| VL | NRLLDGVPSRFSGSGSGTDFTLTISSLQPEDFATYYCQQYDEFPLTFGQGT | |
| | KLEIK | |
| PT1B845 | EVQLVESGGGLVKPGGSLRLSCAASGFTFSSYAMSWVRQAPGKRLEWVASI | 47 |
| VH | SKGGNTYYPNSVKGRFTISRDNAKNSLYLQMNSLRAEDTAVYYCARGWGDY | |
| | GWFAYWGQVTLVTVSS | |
| PT1B845 | DIQMTQSPSSLSASVGDRVTITCKASQDINRYLNWFQQKPGKAPKSLIYRA | 48 |
| VL | NRLLSGVPSRFSGSGSGTDFTLTISSLQPEDMATYYCQQYDEFPLTFGQGT | |
| | KLEIK | |
| PT1B846 | EVQLVESGGGLVKPGGSLRLSCAASGFTFSSYAMSWVRQAPGKRLEWVASI | 49 |
| VH | SKGGNTYYPNSVKGRFTISRDNAKNSLYLQMNSLRAEDTAVYYCARGWGDY | |
| | GWFAYWGQVTLVTVSS | |
| PT1B846 | DIQMTQSPSSLSASVGDRVTITCKASQDINRYLNWFQQKPGKSPKTLIYRA | 50 |
| VL | NRLLSGVPSRFSGSGSGTDFTLTISSLQPEDFATYYCQQYDEFPLTFGQGT | |
| | KLEIK | |
| PT1B847 | EVQLVESGGGLVKPGGSLRLSCAASGFTFSSYAMSWVRQAPGKRLEWVASI | 51 |
| VH | SKGGNTYYPNSVKGRFTISRDNAKNSLYLQMNSLRAEDTAVYYCARGWGDY | |
| | GWFAYWGQVTLVTVSS | |
| PT1B847 | DIQMTQSPSSLSASVGDRVTITCKASQDINRYLNWFQQKPGKAPKTLIYRA | 52 |
| VL | NRLLDGVPSRFSGSGSGTDFTLTISSLQPEDFATYYCQQYDEFPLTFGQGT | |
| | KLEIK | |
| PT1B848 | EVQLVESGGGLVKPGGSLRLSCAASGFTFSSYAMSWVRQAPGKRLEWVASI | 53 |
| VH | SKGGNTYYPDSVKGRFTISRDNAKNSLYLQMNSLRAEDTAVYYCARGWGDY | |
| | GWFAYWGQVTLVTVSS | |
| PT1B848 | DIQMTQSPSSLSASVGDRVTITCKASQDINRYLNWFQQKPGKAPKSLIYRA | 54 |
| VL | NRLLSGVPSRFSGSGSGTDFTLTISSLQPEDMATYYCQQYDEFPLTFGQGT | |
| | KLEIK | |
| PT1B849 | EVQLVESGGGLVKPGGSLRLSCAASGFTFSSYAMSWVRQAPGKRLEWVASI | 55 |
| VH | SKGGNTYYPDSVKGRFTISRDNAKNSLYLQMNSLRAEDTAVYYCARGWGDY | |
| | GWFAYWGQVTLVTVSS | |
| PT1B849 | DIQMTQSPSSLSASVGDRVTITCKASQDINRYLNWFQQKPGKSPKTLIYRA | 56 |
| VL | NRLLSGVPSRFSGSGSGTDFTLTISSLQPEDFATYYCQQYDEFPLTFGQGT | |
| | KLEIK | |
| PT1B850 | EVQLVESGGGLVKPGGSLRLSCAASGFTFSSYAMSWVRQAPGKRLEWVASI | 57 |
| VH | SKGGNTYYPDSVKGRFTISRDNAKNSLYLQMNSLRAEDTAVYYCARGWGDY | |
| | GWFAYWGQVTLVTVSS | |
| PT1B850 | DIQMTQSPSSLSASVGDRVTITCKASQDINRYLNWFQQKPGKAPKTLIYRA | 58 |
| VL | NRLLDGVPSRFSGSGSGTDFTLTISSLQPEDFATYYCQQYDEFPLTFGQGT | |
| | KLEIK | |
| PT1B851 | EVQLVESGGGLVKPGGSLRLSCAASGFTFSSYAMSWVRQAPGKGLEWVASI | 59 |
| VH | SKGGNTYYPNSVKGRFTISRDNAKNSLYLQMNSLRAEDTAVYYCARGWGDY | |
| | GWFAYWGQVTLVTVSS | |
| PT1B851 | DIQMTQSPSSLSASVGDRVTITCKASQDINRYLNWFQQKPGKAPKSLIYRA | 60 |
| VL | NRLLSGVPSRFSGSGSGTDFTLTISSLQPEDMATYYCQQYDEFPLTFGQGT | |
| | KLEIK | |
| PT1B852 | EVQLVESGGGLVKPGGSLRLSCAASGFTFSSYAMSWVRQAPGKGLEWVASI | 61 |
| VH | SKGGNTYYPNSVKGRFTISRDNAKNSLYLQMNSLRAEDTAVYYCARGWGDY | |
| | GWFAYWGQVTLVTVSS | |
| PT1B852 | DIQMTQSPSSLSASVGDRVTITCKASQDINRYLNWFQQKPGKSPKTLIYRA | 62 |
| VL | NRLLSGVPSRFSGSGSGTDFTLTISSLQPEDFATYYCQQYDEFPLTFGQGT | |
| | KLEIK | |
| PT1B853 | EVQLVESGGGLVKPGGSLRLSCAASGFTFSSYAMSWVRQAPGKGLEWVASI | 63 |
| VH | SKGGNTYYPNSVKGRFTISRDNAKNSLYLQMNSLRAEDTAVYYCARGWGDY | |
| | GWFAYWGQVTLVTVSS | |
| PT1B853 | DIQMTQSPSSLSASVGDRVTITCKASQDINRYLNWFQQKPGKAPKTLIYRA | 64 |
| VL | NRLLDGVPSRFSGSGSGTDFTLTISSLQPEDFATYYCQQYDEFPLTFGQGT | |
| | KLEIK | |
| PT1B854 | EVQLVESGGGLVKPGGSLRLSCAASGFTFSSYAMSWVRQNPGKGLEWVASI | 65 |
| VH | SKGGNTYYPDSVKGRFTISRDNAKNSLYLQMNSLRAEDTAVYYCARGWGDY | |
| | GWFAYWGQVTLVTVSS | |
| PT1B854 | DIQMTQSPSSLSASVGDRVTITCKASQDINRYLNWFQQKPGKAPKSLIYRA | 66 |
| VL | NRLLSGVPSRFSGSGSGTDFTLTISSLQPEDMATYYCQQYDEFPLTFGQGT | |
| | KLEIK | |
| PT1B855 | EVQLVESGGGLVKPGGSLRLSCAASGFTFSSYAMSWVRQNPGKGLEWVASI | 67 |
| VH | SKGGNTYYPDSVKGRFTISRDNAKNSLYLQMNSLRAEDTAVYYCARGWGDY | |
| | GWFAYWGQVTLVTVSS | |
| PT1B855 | DIQMTQSPSSLSASVGDRVTITCKASQDINRYLNWFQQKPGKSPKTLIYRA | 68 |
| VL | NRLLSGVPSRFSGSGSGTDFTLTISSLQPEDFATYYCQQYDEFPLTFGQGT | |
| | KLEIK | |
| PT1B856 | EVQLVESGGGLVKPGGSLRLSCAASGFTFSSYAMSWVRQNPGKGLEWVASI | 69 |
| VH | SKGGNTYYPDSVKGRFTISRDNAKNSLYLQMNSLRAEDTAVYYCARGWGDY | |
| | GWFAYWGQVTLVTVSS | |
| PT1B856 | DIQMTQSPSSLSASVGDRVTITCKASQDINRYLNWFQQKPGKAPKTLIYRA | 70 |
| VL | NRLLDGVPSRFSGSGSGTDFTLTISSLQPEDFATYYCQQYDEFPLTFGQGT | |
| | KLEIK | |
| PT1B916 | EVQLVESGGGLVKPGGSLRLSCAASGFTFSSYAMSWVRQAPGKGLEWVASI | 71 |
| VH | SKGGNTYYPDSVKGRFTISRDNAKNSLYLQMNSLRAEDTAVYYCARGWGDY | |
| | GWFAYWGQVTLVTVSS | |
| PT1B916 | DIQMTQSPSSLSASVGDRVTITCKASQDINRYLNWFQQKPGKAPKTLIYRA | 72 |
| VL | NRLLDGVPSRFSGSGSGTDFTLTISSLQPEDFATYYCQQYDEFPLTFGQGT | |
| | KLEIK | |

CDR sequences are underlined

### ELISA

Binding to a synthetic pT217+tau calibrant peptide (PRQEFEVMEDHAGTYGL GDR(dPEG4)GKTKIATPRGAAPPGQKG(dPEG4)GSRSR(pT)PSLP(pT)PPTREPKKV-amide) (SEQ ID NO:81-83, respectively; the full-length sequence is disclosed as SEQ ID NO:79) was analyzed by ELISA. The phosphopeptide (10 ng/mL) was directly coated to the plate, and after blocking with 0.1% casein, incubated with different concentrations of the indicated antibodies (PT3, PT1B296, and the humanized antibodies PT1B844, PT1B847, PT1B850, and PT1B856) expressed as human IgG1. Detection of immunocomplexes was performed by adding an HRPO-labelled Goat F(ab')2 anti-human IgG. After washing, detection was performed using one-step TMB substrate (ThermoScientific; Waltham, Mass.) according to the manufacturer's instructions. Binding data in FIG. 3 showed a drastic reduction in maximal signal for the PT1B296 antibody compared to PT3. This was not observed for the humanized antibodies PT1B844, PT1B847, PT1B850, and PT1B856 which displayed a very similar binding compared to PT3.

Binding Assessment by Surface Plasmon Resonance (SPR)/SPR on Different Phosphor Peptides

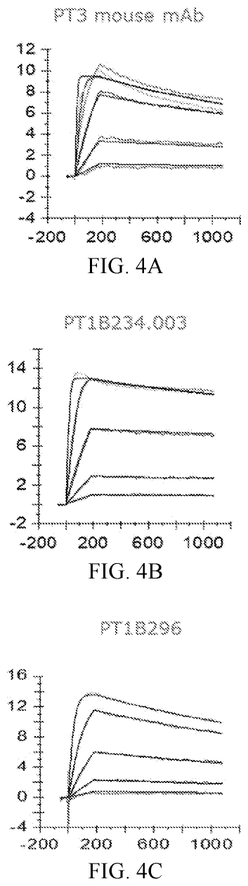

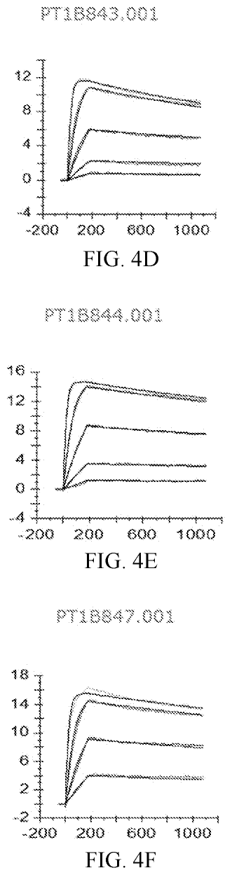

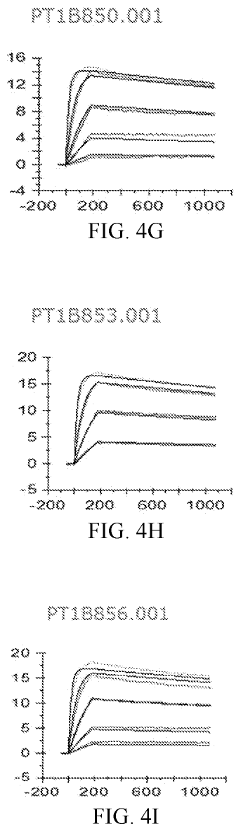

A selected panel of anti-Tau antibodies were tested for binding to tau peptides phosphorylated at different positions. SPR experiments were performed on a Biacore T200 instrument. Briefly, the CM5 sensor chip surface was activated using a 1:1 mixture of EDC/NHS solution. This was followed by covalent immobilization of a mixture of anti-human and anti-mouse Fc-specific IgG solution (>9000 RU) and deactivation of sensor chip surface with ethanolamine. The test antibodies and parent mAb were captured through anti-Fc surface (250-550 RU) and was followed by the injection of serially diluted phosphorylated tau peptides (30 nM-0.37 nM at 3-fold). The association and dissociation were monitored for 3 minutes and 30 minutes respectively. The sensorgrams for the binding of test mAbs to different phosphorylated peptides were analyzed using 1:1 Langmuir model and the results are reported as the on-rates (kₒₙ), off-rates (k_{off}), and binding affinities (K_{D}). The binding sensorgrams for the test antibody panel binding to NPT-6 peptide are shown in FIGS. 4A-4I and the corresponding kinetics and affinities in Table 4. The binding affinities of the test antibodies with the full panel of phosphorylated tau peptides are listed in Table 5, and the phosphorylated tau peptides are shown in Table 6. The test mAbs and the parent mAb show highest affinity (sub-nM range) to the peptides phosphorylated at positions 212 and 217 (NPT-6) and with slightly reduced binding with additional phosphorylation sites in conjunction with 212 and 217 (NPT-5). The absence of phosphorylation at either of the residues (212 or 217) resulted in a significant loss in binding (>μM; NPT-2, NPT-8, PT25-7, PT25-8) and complete loss of binding for peptides without phosphorylated 212 and 217 residues (PT25-6, NPT-C).

TABLE 4
Biacore SPR results for humanized mAbs binding to the high affinity NPT-6 peptide

| | | | 95% | | 95% | AVG | 95% |
|---|---|---|---|---|---|---|---|
| | | AVG ka | CI ka | AVG kd | CI kd | KD | CI KD |
| Sample | Description | (1/Ms) | (1/Ms) | (1/s) | (1/s) | (pM) | (pM) |
| PT1B17.005 | PT3 mG2a | 5.14E+06 | (3.95- | 4.23E−04 | (2.43- | 81.9 | (65.0-98.9) |
| | parent mouse | | 6.32)E+06 | | 6.03)E−04 | | |
| | mAb | | | | | | |
| PT1B296.004 | HFA mAb | 1.33E+06 | (1.10- | 3.33E−04 | (2.28- | 253.7 | (129.9-377.5) |
| | (clinical mAb) | | 1.56)E+06 | | 4.39)E−04 | | |
| PT1B234.003 | PT3 | 3.29E+06 | (1.72- | 1.90E−04 | (1.36- | 72.9 | (25.8-119.9) |
| | control | | 4.86)E+06 | | 2.45)E−04 | | |
| PT1B843.001 | PT3 | 2.07E+06 | (1.53- | 3.68E−04 | (2.60- | 180.4 | (86.9-273.9) |
| | humanized | | 2.61)E+06 | | 4.76)E−04 | | |
| PT1B844.001 | PT3 | 1.99E+06 | (1.40- | 1.55E−04 | (0.558- | 77.2 | (48.5-105.9) |
| | humanized | | 2.57)E+06 | | 2.54)E−04 | | |
| PT1B846.001 | PT3 | 1.33E+06 | (1.20- | 3.19E−04 | (1.05- | 238.0 | (92.4-383.6) |
| | humanized | | 1.47)E+06 | | 5.32)E−04 | | |
| PT1B847.001 | PT3 | 1.60E+06 | (1.35- | 1.15E−04 | (0.189- | 73.3 | (1.1-145.5) |
| | humanized | | 1.85)E+06 | | 2.11)E−04 | | |
| PT1B850.001 | PT3 | 1.75E+06 | (1.48- | 1.27E−04 | (0.432- | 73.2 | (17.5-128.9) |
| | humanized | | 2.01)E+06 | | 2.10)E−04 | | |
| PT1B853.001 | PT3 | 1.32E+06 | (0.979- | 2.23E−04 | (0.827- | 173.3 | (26.7-319.9) |
| | humanized | | 1.65)E+06 | | 3.63)E−04 | | |
| PT1B856.001 | PT3 | 1.64E+06 | (1.47- | 1.85E−04 | (0.829- | 113.6 | (43.0-184.2) |
| | humanized | | 1.81)E+06 | | 2.87)E−04 | | |

TABLE 5
Biacore SPR results for humanized mAbs binding to phosphorylated tau peptides
Phospho-peptides K_{D} (pM)

| Sample | NPT-6 | NPT-2 | NPT-5 | NPT-8 | PT25-7 | PT25-8 | PT25-6 | NPT-C |
|---|---|---|---|---|---|---|---|---|
| PT1B234.003 | 92 | 16,933 | 236 | 12,100 | 8,793 | 7,860 | Low/no | Low/no |
| | | | | | | | binding | binding |
| PT1B844.001 | 89 | 15,011 | 140 | 8,356 | 11,411 | 6,911 | Low/no | Low/no |
| | | | | | | | binding | binding |
| PT1B847.001 | 73 | 21,567 | 277 | 18,167 | 13,233 | 11,233 | Low/no | Low/no |
| | | | | | | | binding | binding |
| PT1B850.001 | 73 | 20,900 | 315 | 15,367 | 16,000 | 10,047 | Low/no | Low/no |
| | | | | | | | binding | binding |
| PT1B856.001 | 114 | 19,900 | 250 | 12,867 | 10,297 | 8,497 | Low/no | Low/no |
| | | | | | | | binding | binding |
| PT1B296.004 | 354 | 58,333 | 429 | 48,500 | 39,067 | 53,633 | Low/no | Low/no |
| | | | | | | | binding | binding |
| PT1B333.005 | 337 | 33,967 | 414 | 21,300 | 29,067 | 26,933 | Low/no | Low/no |
| | | | | | | | binding | binding |

TABLE 6
Peptide Sequences

| Peptide | 204 | | | | | | 210 | | 212 | | 214 | | | 217 | | | 220 | | | | | 225 |
|---|---|---|---|---|---|---|---|---|---|---|---|---|---|---|---|---|---|---|---|---|---|---|
| NPT-6 | G | T | P | G | S | R | S | R | T* | P | S | L | P | T* | P | P | T | R | E | P | K | K |
| NPT-2 | G | T | P | G | S | R | S | R | T* | P | S | L | P | T | P | P | T | R | E | P | K | K |
| NPT-5 | G | T | P | G | S | R | S | R | T* | P | S* | L | P | T* | P | P | T | R | E | P | K | K |
| NPT-8 | G | T | P | G | S | R | S | R | T* | P | S* | L | P | T | P | P | T | R | E | P | K | K |
| PT25-7 | G | T | P | G | S | R | S | R | T | P | S | L | P | T* | P | P | T | R | E | P | K | K |
| PT25-8 | G | T | P | G | S | R | S | R | T | P | S* | L | P | T* | P | P | T | R | E | P | K | K |
| PT25-6 | G | T | P | G | S | R | S | R | T | P | S* | L | P | T | P | P | T | R | E | P | K | K |
| NPT-C | G | T | P | G | S | R | S | R | T | P | S | L | P | T | P | P | T | R | E | P | K | K |

*phosphorylated residue

### SPR on PHF (Fab+mAb)

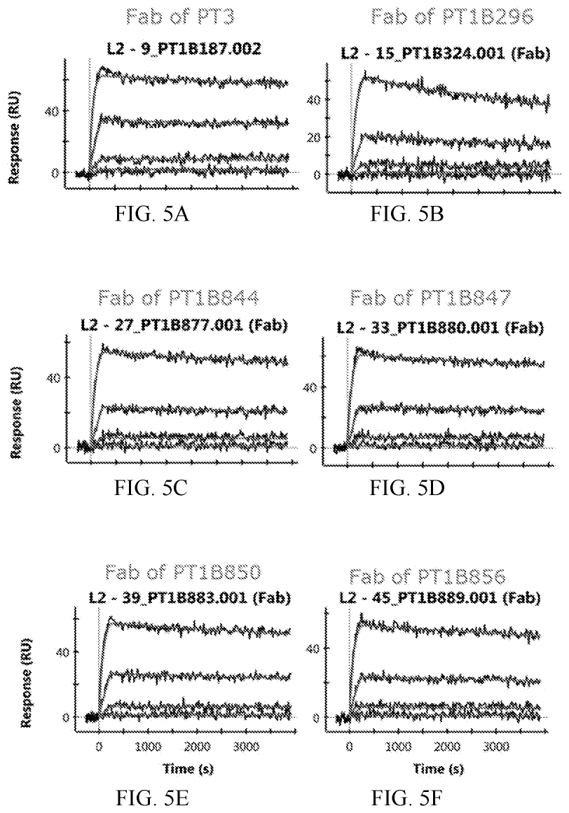

The purified Fabs of a selected panel of test mAbs were tested for binding to the patient-derived PHF-Tau material to determine their intrinsic monovalent affinities. The interaction of anti-tau Fabs with PHF-tau was analyzed by ProteOn using a biosensor surface prepared by capture-coupling PHF-tau through HT7 mouse mAb as a capture reagent. Briefly, a GLC sensor chip was activated using a 1:1 mixture of sulfo-NHS/EDC solution. HT7 mAb was covalently immobilized to the surface of the sensor chip using amine-coupling chemistry (>3000 RU) and the surface was deactivated with ethanolamine. This was followed by capture-coupling of 2× centrifuged PHF-Tau over the HT7 surface (>200 RU) and the injection of ethanolamine to block any remaining reactive esters. The anti-tau Fabs were diluted in the running buffer (HBS with 0.05% Tween and 3 mM EDTA) and injected in solution (0.02-3 nM in 5-fold dilutions). The association and dissociation were monitored for 4 and 60 minutes, respectively. Regeneration of the sensor surface was performed using 10 mM Gly pH 2.0. The sensorgrams for Fab-PHF-Tau interactions were analyzed using a 1:1 Langmuir binding model and the results are reported as on-rates (kₒₙ), off-rates (k_{off}), and binding affinities (K_{D}). All Fabs bind very tightly to PHF-Tau with affinities in the low-pM range. The humanized Fabs retain similar affinities as the parent Fab, PT1B187 (FIGS. 5A-5F and Table 7).

TABLE 7
ProteOn SPR results for humanized Fabs binding to PHF-Tau

| | Corre- | kₒₙ ± 95% | k_{off} ± 95% | K_{D} ± 95% |
|---|---|---|---|---|
| | sponding | C.I | C.I | C.I |
| Fab | mAb | (×10⁶ 1/Ms) | (×10⁻⁵ 1/s) | (pM) |
| PT1B187 (PT3) | PT3 | 5.1 ± 0.3 | 3.3 ± 0.6 | 6.6 ± 1.7 |
| PT1B324 (B296) | JNJ′657 | 3.0 ± 0.3 | 8.8 ± 0.2 | 29.7 ± 3.0 |
| PT1B877 (B844) | PT1B844 | 3.3 ± 0.4 | 3.2 ± 0.4 | 9.7 ± 2.1 |
| PT1B880 (B847) | PT1B847 | 3.8 ± 0.4 | 3.1 ± 0.5 | 8.4 ± 2.0 |
| PT1B883 (B850) | PT1B850 | 3.9 ± 0.2 | 3.3 ± 0.5 | 8.5 ± 1.8 |
| PT1B889 (B856) | PT1B856 | 3.6 ± 0.4 | 3.3 ± 0.6 | 9.4 ± 2.5 |

### Materials and Methods for HDX-MS Paratope Mapping

On-Exchange Experiment for HDX-MS. On-exchange reaction was initiated by mixing 4 μL of 25 μM Tau Fab (PT1B187 or PT1B887) with or without 30 μM NPT-6 and 36 μL of H₂O or a deuterated buffer (20 mM MES, pH 6.4, 150 mM NaCl in 95% D₂O or 20 mM Tris, pH 8.4, 150 mM NaCl in 95% D₂O). The reaction mixture was incubated for 15, 50, 150, 500, 1,500, 5,000 or 15,000 seconds at 3.2° C. or 23° C. The on-exchanged solution was quenched by the addition of 40 μL of chilled 8 M urea, 1 M TCEP, pH 3.0 and immediately analyzed.

General Procedure for HDX-MS Data Acquisition. HDX-MS sample preparation was performed with automated HDx system (LEAP Technologies, Morrisville, N.C.). The columns and pump were; protease, protease type XIII (protease from Aspergillus saitoi, type XIII)/pepsin column (w/w, 1:1; 2.1×30 mm) (NovaBioAssays Inc., Woburn, Mass.); trap, ACQUITY UPLC BEH C18 VanGuard Pre-column (2.1×5 mm) (Waters, Milford, Mass.), analytical, Accucore C18 (2.1×100 mm) (Thermo Fisher Scientific, Waltham, Mass.); and LC pump, VH-P10-A (Thermo Fisher Scientific). The loading pump (from the protease column to the trap column) was set at 600 μL/min with 99% water, 1% acetonitrile, 0.1% formic acid. The gradient pump (from the trap column to the analytical column) was set from 8% to 28% acetonitrile in 0.1% aqueous formic acid in 20 min at 100 μL/min.

MS Data Acquisition. Mass spectrometric analyses were carried out using an LTQ™ Orbitrap Fusion Lumos mass spectrometer (Thermo Fisher Scientific) with the capillary temperature at 275° C., resolution 150,000, and mass range (m/z) 300-2,000.

HDX-MS Data Extraction. BioPharma Finder 3.0 (Thermo Fisher Scientific) was used for the peptide identification of non-deuterated samples prior to the HDX experiments. HDExaminer version 2.1 (Sierra Analytics, Modesto, Calif.) was used to extract centroid values from the MS raw data files for the HDX experiments.

### Results

Segments 30-32, 32-33 (CDR1), 100-101, 102, 103-105, 107 (CDR3) of heavy chain of PT1B187 (Corresponding mAb is PT3) were very strongly perturbed upon binding to NPT-6, indicating they are important parts of the paratope. Segment 93-96 (CDR3) of light chain was also significantly perturbed and is involved in the paratope. Segment 50-53 (CDR2) of light chain was likely to be involved in the binding. Although the average change in segment 50-53 was small, it was clear from the deuterium buildup curves that this segment will show significant perturbation if longer time points were monitored. CDR2 of heavy chain and CDR1 of light chain were not likely to be a part of paratope. Some parts of heavy chain, segments 20-23, 44-45, 69, 72-73, 75-77, and 78, showed significant change in deuteration levels upon binding to NPT-6, presumably due to allosteric effects.

Segments 30-31, 32 (CDR1), 53-54 (CDR2) 97-98, 99, 100-101, 102, 103-104, and 105 (CDR3) of heavy chain of PT1B877 (Corresponding mAb is PT1B844) were very strongly perturbed upon binding to NPT-6, indicating all three CDRs in heavy chain were important parts of the paratope. CDR2 of light chain may be involved in the binding. Although the average change in segment 50-55 was just under the threshold of 10%, it was clear from the deuterium buildup curves that this segment would show significant perturbation if longer time points were monitored. CDR1 (residues 27-32) of light chain was not likely to be a part of the paratope. CDR3 of the light chain was not monitored by HDX-MS. Some parts of heavy chain, segments 22, 44-45, 69, 70-71, and 74-78, showed significant change in deuteration levels upon binding to NPT-6, presumably due to allosteric effects.

### Example 2

### Evaluation of Reactivity with Aggregated and Non-Aggregated Tau

Species Cross Reactivity with Western Blotting

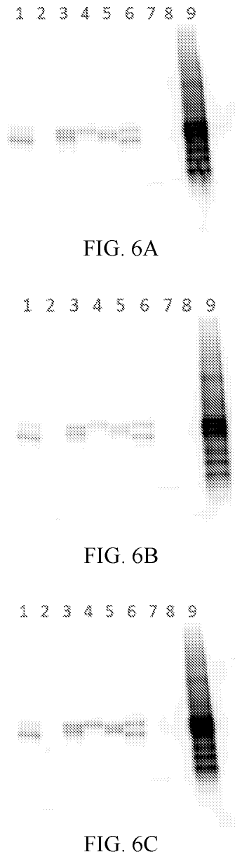

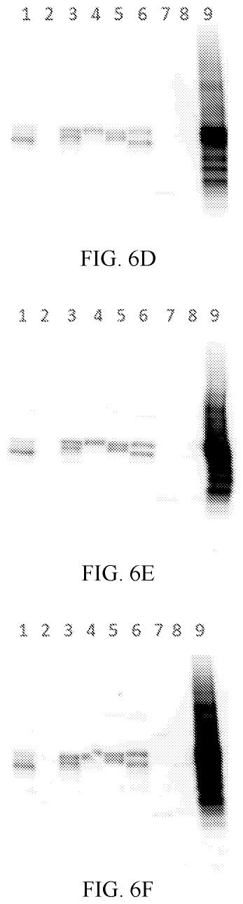

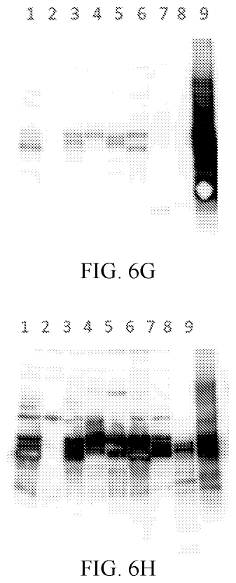

Further profiling of antibodies was performed by evaluating the binding to tau in brain samples from different species (mouse, rat, dog, minipig, marmoset, cynomolgous monkey, and human). For human tau, a distinction was made between soluble Tau (heat-stable extract from non-AD human brain) and aggregated PHF Tau (sarcosyl insoluble prep from human AD brain (Braak VI)). To be able to detect lower affinity interactions to non-Tau related proteins, antibodies were tested at a concentration of 1 μg/mL. Relatively high amounts of brain homogenates (20 μg of total protein) were loaded on the gel (4-12% Bis-Tris Precast gels (BioRad; Hercules, Calif.), and the run was performed in MOPS buffer (3-morpholinopropane-1-sulfonic acid). After the run, proteins were transferred to a nitrocellulose membrane by dry-blotting using Turboblot system (BioRad). After the transfer, membranes were blocked using 5% Non-fat dry milk dissolved in Tris-buffered saline Tween (TBS-T) for at least 1 hour and incubated overnight (4° C.) in the presence of antibodies PT3, PT1B296, and the humanized antibodies PT1B844, PT1B847, PT1B850, and PT1B856 (all at 1 μg/mL), or for 1 hour with HRPO labelled PT9 (Vandermeeren et al., J. Alzheimers Dis. 65(1):265-281 (2018)). After washing (3×5 minutes in TBS-T), the primary antibodies were detected with an HRPO-labelled goat anti human IgG (Jackson Immunoresearch; West Grove, Pa.) for 1 hour. After the final washing step (4×5 minutes in TBS-T), detection was performed using ECL West Dura (Thermo Scientific), and the images were scanned. An overview of these profiles is shown in FIGS. 6A-6H and clearly indicated reactivity of all PT3 variants with tau from mouse, rat, dog, minipig, and marmoset brain.

Binding to tau in a human HSE was only very minor in comparison to the strong reactivity to AD brain-derived PHF. As expected, none of the antibodies reacted with the brain extract from tau KO mice. Additionally, none of the antibodies reached with tau from Cynomolgous monkey from which the threonine at position 220 is mutated into an alanine. Overall, the data confirmed that the humanization process did not alter the reactivity towards tau from different species.

Antibody Profiling using IHC

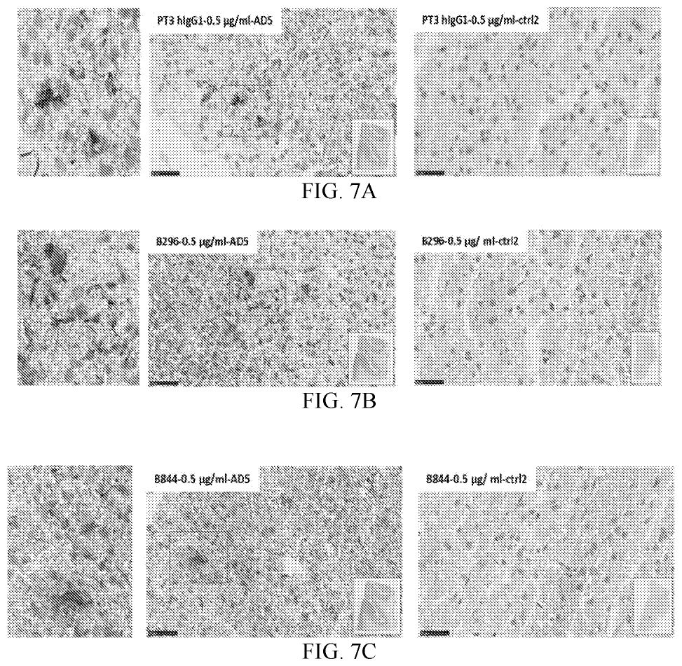

Immunohistochemical analysis was performed on cryosections of AD and non-AD brain to confirm reactivity with physiological and pathophysiological tau in situ. Cryopreserved human brain tissue was sliced with a cryostat (20 μm thickness) and stored at −80° C. before use. Sections were dried, followed by formalin fixation, blocking of endogenous peroxidase with 3% hydrogen peroxide (DAKO, Glostrup, Denmark, S2023), and permeabilization in PBS1x+0.3% Triton X-100 for 1 hour. Primary antibodies (0.4 μg/ml) were diluted in antibody diluent with background reducing components (DAKO, S3022) and applied to the sections for 1 hour. After extensive washing, slides were incubated with HRP-conjugated anti-mouse secondary antibody (Envision, DAKO, K4000), followed by chromogenic DAB labelling (DAKO, K4368). Slides were counterstained with hematoxylin, dehydrated, and mounted with organic mounting medium (Vectamount, Vector labs, Burlingame, Calif., USA, H-5000). Imaging was performed with a Hamamatsu NanoZoomer 2.0 rs (Hamamatsu Photonics, Shizuoka, Japan). PT3, PT1B296, and the humanized antibody PT1B844 showed similar (strong) binding to aggregated tau from AD brain and only minor to no reactivity to similar section derived from non-AD brain (FIGS. 7A-7C), which is consistent with the Western blotting data (FIGS. 6A-6H).

### Example 3

### Functional Testing in Cellular Assays

PT3, PT1B296, and the humanized antibodies PT1B844, PT1B847, PT1B850, and PT1B856 were tested for inhibition of tau seeding in an immune-depletion assay. The assay utilizes HEK cells expressing two chromophore-tagged K18 tau fragments that generate a signal when in close proximity by aggregation. When the cells were treated with seeds of aggregated and phosphorylated full-size tau derived from different sources, a K18 aggregate was induced that was quantified by counting fluorescence resonance energy transfer (FRET)-positive cells using fluorescence-activated cell sorting (FACS) (Holmes et al.,2014, PNAS. 111(41):E4376-85).

Immunodepletion Cellular Assays

To investigate if the maximum percentage inhibition value is related to the density of epitopes on the seeds or to the number of seeds that contain the epitope for PT3, PT1B296, and the humanized antibodies PT1B844, PT1B847, PT1B850 and PT1B856, immunodepletion assays were performed. In the immunodepletion assays, the tau seeds were incubated with test antibody and removed from the solution with protein G beads. The depleted supernatant was tested for residual seeding capacity in the chromophore-K18-containing HEK cells and analyzed by FACS as previously described (Holmes et al., Proc Natl Acad Sci U S A. 111(41):E4376-85, 2014).

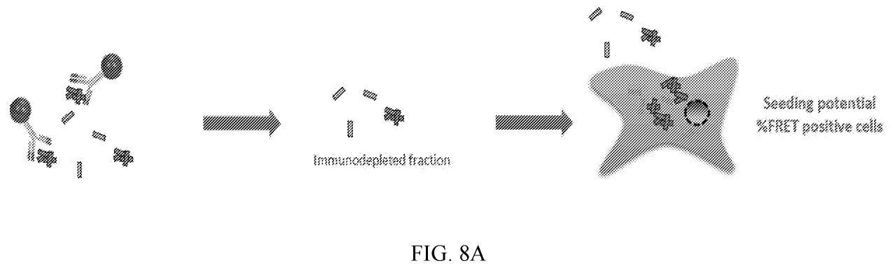

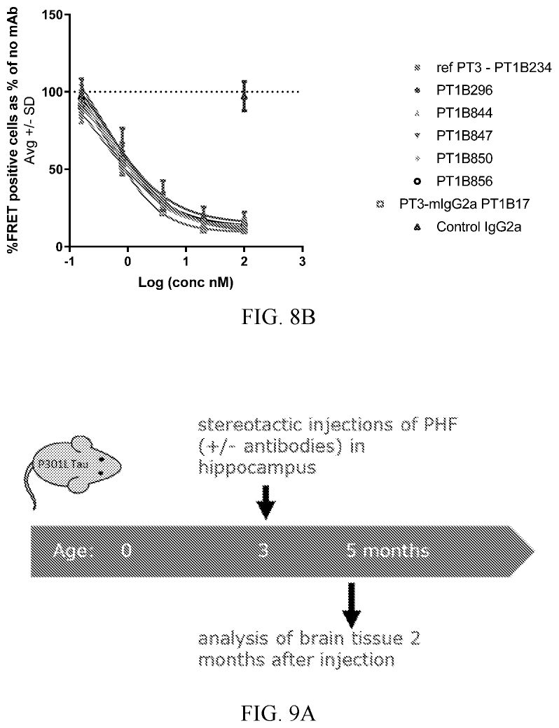

Homogenates containing tau seeds for immunodepletion were generated from cryopreserved human AD brain tissue. In the human AD brain immunodepletion assay, the supernatant after depletion was tested in the presence of the transfection reagent Lipofectamine2000 to obtain an acceptable assay window. The tau seeding could be reduced completely with PT3, PT1B296, and the humanized antibodies PT1B844, PT1B847, PT1B850, and PT1B856 (FIGS. 8A and 8B) in total homogenates from human AD brain. As a result of the complex constitution of the total homogenate (which contains both monomeric (non-seeding) tau and seeding tau fibrils), differences in antibody affinity are not picked up in this type of assay.

The mechanism of action for tau antibody therapy is still a matter of debate and multiple mechanisms have been proposed. Antibody-mediated clearance of extracellular seeds by microglial cells has recently been suggested as one dominant mechanism of action (Funk et al., J Biol Chem. 290(35):21652-62, 2015 and McEwan et al., 2017, PNAS 114:574-9). In this context, immunodepletion of human-brain-derived seeding material can be considered the most translational cellular result, and the high maximal efficacy of the humanized antibodies, which was similar to the efficacy of PT3, suggests that current humanized molecules PT1B844, PT1B847, PT1B850, and PT1B856 are promising therapeutic candidates.

### Example 4

### In Vivo Efficacy in a PHF Injection Model

A transgenic P301L mouse injection model has been established, wherein a pro-aggregating fragment of tau, such as synthetic K18 fibrils (Li and Lee, Biochemistry. 45(51):15692-701, 2006) or PFH-tau seeds derived from human AD brain, was injected in cortical or hippocampal regions of P301L transgenic mouse models at an age at which cell-autonomous aggregation had not started. The injection model aimed to mimic the critical extracellular seeding component of tau spreading. The injected K18 or PHF-tau seed induced tauopathy at the injection site and, to a lesser degree, at the connected contralateral region (Peeraer et al., Neurobiol Dis. 73:83-95, 2015). The model enabled testing of the anti-seeding potential of antibodies, such as anti-tau antibodies of the invention, when co-injected with the AD-brain-derived PHF-tau seeds or the K18 fibrils (Iba et al., 2015, J Neurosci. 33(3):1024-37, 2013; Iba et al., Acta Neuropathol. 130(3):349-62).

Cortical injection of a sarcosyl-insoluble fraction of post-mortem AD brain triggered a slowly progressing increase of tau aggregation. In the injected hemisphere, the first signals were measured 1 month after injection and progress further 3 months after injection. Five months after injection, some animals started to form tangles driven by the P301L mutation (Terwel et al., J Biol Chem. 280(5):3963-73, 2005). AT8 staining levels increased between 1 and 3 months (US Pat. Publication No. 2018/0265575), so antibody efficacy experiments were analyzed 2 months after co-injection (Vandermeeren et al., J. Alzheimers Dis. 65(1): 265-81 (2018)). Additionally, hippocampal injection of a sarcosyl-insoluble fraction of post-mortem AD brain triggered a dose-dependent increase of tau aggregation measured by

MesoScale Discoveries (MSD; Meso Scale Discovery; Rockville, Md.) analysis of sarcosyl insoluble fractions from the injected hemispheres.

Animal Treatment and Intracranial Injections

For injection studies, transgenic tau-P301L mice, expressing the longest human tau isoform with the P301L mutation (tau-4R/2N-P301L) (Terwel et al., 2005, Id.) were used for surgery at the age of 3 months. All experiments were performed in compliance with protocols approved by the local ethical committee. For stereotactic surgery, the mice received a unilateral (right hemisphere) injection in the hippocampus (AP -2.0, ML +2.0 (from bregma), DV 1.8 mm (from dura)) of 3 μl (speed 0.25 μl/min) with a sarcosyl insoluble prep from postmortem AD tissue (enriched paired helical filaments, ePHF) in the presence or absence of monoclonal antibodies. Mice were sacrificed for dissection (2 months after intracranial injection).

Extraction Procedure

Mouse tissue from the injected hemisphere was weighed and homogenized in 6 volumes of homogenization buffer (10 mM Tris HCl (pH7.6); 0.8 M NaCl; 10% w/v sucrose; 1 mM EGTA; PhosStop phosphatase inhibitor cocktail; complete EDTA-free mini protease inhibitors). The homogenate was centrifuged at 28,000×g for 20 minutes, and 1% N-laurylsarcosine was added after taking an aliquot from the resulting supernatant (total homogenate). After 90 minutes (900 rpm, 37° C.), the solutions were again centrifuged at 184,000×g for 1 hour. The supernatants were kept as a sarcosyl-soluble fraction, whereas the pellet containing the sarcosyl-insoluble material was resuspended in homogenization buffer.

Biochemical Analysis

Coating antibody (PT51) was diluted in PBS (1 μg/ml) and aliquoted into MSD plates (30 μL per well) (L15XA, Mesoscale Discoveries), which were incubated overnight at 4° C. After washing with 5×200 μl of PBS/0.5%Tween-20, the plates were blocked with 0.1% casein in PBS and washed again with 5×200 μl of PBS/0.5%Tween-20. After adding samples (sarcosyl insoluble fractions) and standards (both diluted in 0.1% casein in PBS), the plates were incubated overnight at 4° C. Subsequently, the plates were washed with 5×200 μl of PBS/0.5%Tween-20, and SULFO-TAG™ conjugated detection antibody (PT51) in 0.1% casein in PBS was added and incubated for 2 hours at room temperature while shaking at 600 rpm. After a final wash (5×200 μl of PBS/0.5%Tween-20), 150 μl of 2× buffer T was added, and plates were read with an MSD imager. Raw signals were normalized against a standard curve consisting of 16 dilutions of a sarcosyl insoluble prep from postmortem AD brain (ePHF). The raw signals and standard curve were expressed as arbitrary units (AU) ePHF. Statistical analysis (ANOVA with Bonferroni post-test) was performed with the GraphPad prism software and with an ‘in house’ developed application for automated analysis.

Results

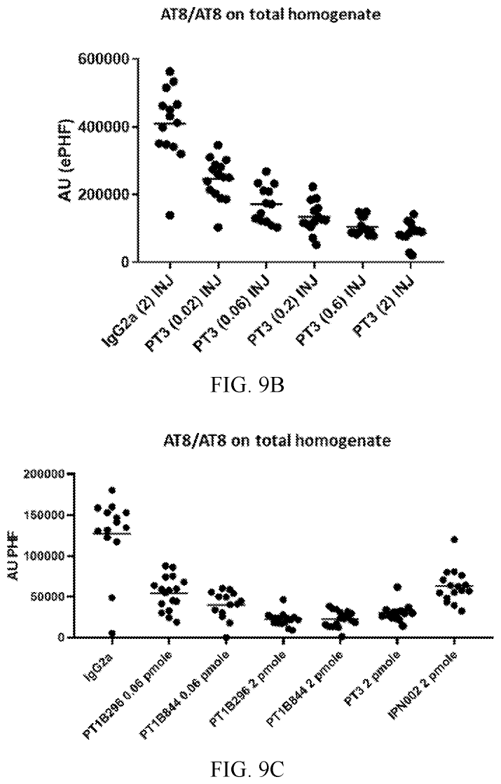

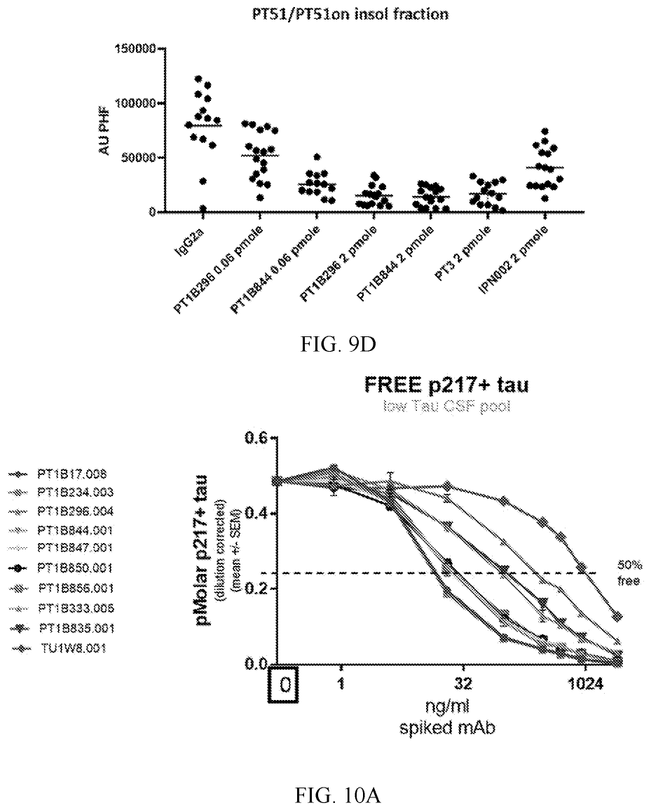

Several of the internal anti-Tau antibodies and of PT3 had been evaluated in this co-injection model (see, e.g., U.S. Pat. Pub. No. 2018/0265575 and Vandermeeren et al., J. Alzheimers Dis. 65(1):265-81 (2018)). In this study, all molecules were tested as chimeric mG2a variants. From the humanized variants, PT1B844 was compared to PT3, PT1B296, and to IPN002 in a co-injection model where 2 pmole of antibody is co-injected with 2 pmole of PHF as described in Vandermeeren et al., J. Alzheimers Dis. 65(1):265-81 (2018)). Data in FIGS. 9B-9D demonstrate that this amount exerts the maximal efficacy by the antibody. For PT3, PT1B296, and PT1B844, which was clearly more outspoken compared to the effect by IPN002. In addition to that concentration, a lower antibody concentration (0.06 pmole) was used for the comparison between PT1B296 and PT1B844.

### Example 5

### Ex Vivo Epitope Engagement

Although pathological tau seeds are difficult to detect, different tau fragments have been detected in cerebrospinal fluid (CSF). Use of ultrasensitive SIMOA technology allowed for the detection of the epitope in CSF utilizing the PT3, PT1B296, and the humanized antibodies (see, e.g., U.S. Pat. Pub. No. 2019/0271710). Antibody treatment resulted in a reduction of free pT217+ signals (see, e.g., Galpern et al., Alzheimer's & Dementia: The Journal of the Alzheimer's Association 15(7):252-3 (2019))). Since the humanized antibodies have similar affinity compared to PT3 and a higher affinity compared to PT1B296, it was expected that differences in affinity were reflected in different potencies in a CSF spike assay where increasing antibody concentrations were “spiked” into CSF after which the free pT217+ levels were measured with SIMOA as described in (see, e.g., U.S. Pat. Pub. No. 2019/0271710).

M&M for SIMOA

Assay-specific reagents were as follows: Simoa Homebrew kit (Quanterix, cat# 101351; Quanterix; Billerica, Mass.), Helper beads (Quanterix, cat# 101732), pT3 mouse monoclonal antibody (mAb), hT43 mAb, pT82 mAb and hT7 mAb. pT3 is the parental antibody that recognizes p217+ tau, and the humanized version thereof is referred to herein as humanized pT3 mAb.

The samples were diluted in 50 mM Tris, 50 mM NaCl, 5 mM EDTA, 2% Bovine Serum Albumin, 0.1% Tween 20, 0.05% ProClin 300, pH7.8.

Two custom peptides made by New England Peptide (Gardner, Mass.) were used to calibrate the assay (calibrant peptides).

Peptide pT3xhT43 contains hT43, PT51 and pT3 epitopes connected by PEG4 linkers and has a molecular weight of 6893 g/mol. The amino acid sequence of peptide pT3xhT43 is PRQEFEVMEDHAGTYGLGDR(dPEG4)GKTKIATPRGAAPPGQKG(dPEG4)GSRSR(pT) PSLP(pT)PPTREPKKV-amide (SEQ ID NO:81-83, respectively; the full-length sequence is disclosed as SEQ ID NO:79).

Peptide pT3xpT82 contains pT82 and pT3 epitopes connected by a PEG4 linker and has a molecular weight of 4551 g/mol. The amino acid sequence of peptide pT3xpT82 is Ac-SLEDEAAGHVTQARMVSK(dPEG4)GSRSR(pT)PSLP(pT)PPTREPKKV-amide (SEQ ID NO:84 and 83, respectively; the full-length sequence is disclosed as SEQ ID NO:80).

Reagent Prep

The capture beads were coated with 0.3 mg/ml capture Ab following the protocol provided in the Quanterix manual. The coated capture beads were diluted in Bead Diluent Buffer to 200,000 beads/ml, and 200,000 beads/ml Helper Beads were added so that the total concentration of beads was 400,000 beads/ml. The detection antibodies were biotinylated at 60× following the protocol provided in the Quanterix manual and were diluted in Homebrew Detector/Sample Diluent to 1.8 μg/ml.

The calibrant peptides were reconstituted to 5 mg/ml in 0.1% phosphoric acid/water, aliquoted to 20 μl and frozen. When ready for use, the calibrant peptide aliquots were thawed and diluted 1:1000 (e.g. 1.5 μl into 1498.5 μl), and the dilutions were diluted 1:1000 so that the final concentration of the peptides was 5000 pg/ml. A standard curve with 3× jumps was made, starting at 30 pg/ml.

CSF samples were diluted at least 1:4 in Sample Diluent. Healthy volunteer (HV) samples were diluted 1:5 or 1:10, and AD samples were diluted at least 1:20.

Simoa Assay

A custom Simoa assay was created comprising a two-step protocol comprising 35 minutes with capture Ab, sample, and detection Ab, and washing, followed by 5 minutes with streptavidin β-galactosidase (SBG). Each reaction comprised 25 μl beads solution, 100 μl sample or calibrant, 20 μl detection solution, 100 μl SBG. The antibodies were assigned names, and up to five capture antibodies and five detection antibodies could be loaded at a time. The reactions were performed in the Simoa cuvettes by the instrument, washed one last time, and loaded into measurement discs with β-galactosidase substrate (RGP) before measurements were taken by the instrument.

Results

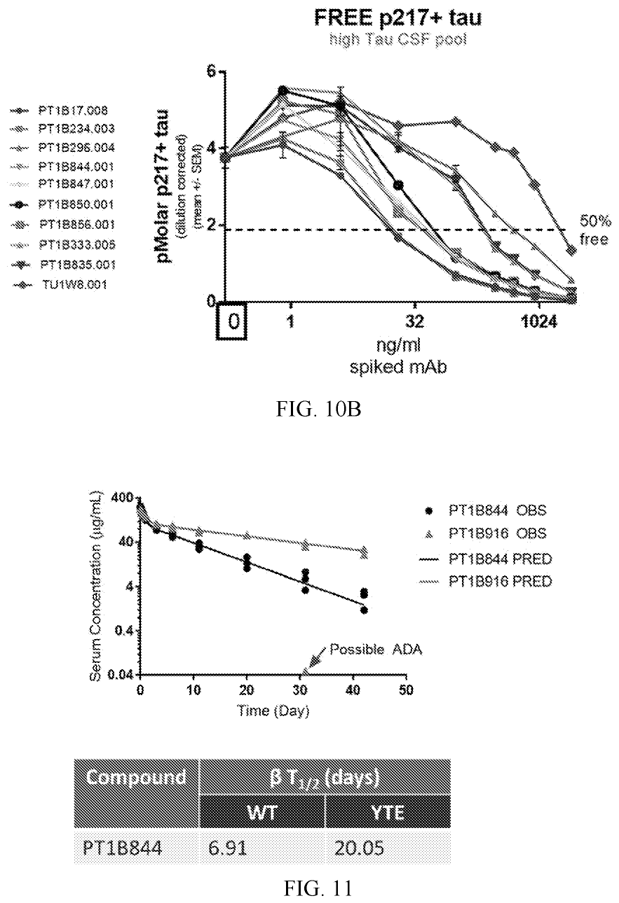

Data in FIGS. 10A-10B showed a concentration dependent reduction of the pT217+ signal in a CSF pool with low levels of pT217+ but also in a pool containing high pT217+ levels measured by SIMOA. Consistent with the peptide affinity data, a more potent inhibition was observed for PT3 (EC₅₀ value 14 ng/mL) and the humanized variants (including PT1B844 (EC₅₀ value 24 ng/mL)) compared to PT1B296 (EC50 value 248 ng/mL).

### Example 6

### Improved Plasma T½ for PT1B916, the YTE Variant of PT1B844.

An important parameter for the therapeutic potential of an antibody, is its plasma half-life. As this can be increased by modifying FcRn affinity through M252Y/S254T/T256E mutations in the Fc-region (Dall'Acqua et al., JBC 281(33):23514-24 (2006)), this strategy was applied to the PT1B844 molecule to generate PT1B916, a molecule with identical antigen binding region but with M252Y/S254T/T256E mutation in the Fc region. To demonstrate its plasma half-life, a single dose plasma PK study was performed in Cynomolgus. After IV injection of PT1B844 or PT1B916 (10 mg/kg), plasma samples were collected at the time points indicated in FIG. 11 with the last sample taken at day 42 after dosing.

Allotype-Selective Detection of Human IgG1 mAbs.

Data in FIG. 11 showed a time-dependent reduction of antibody levels in plasma. The decay of the PT1B916 (20.05 days +/−) occurred slower in comparison to PT1B844 (6.95 days +/−, which indicated a 2.5-fold increase in plasma half-life for PT1B916.

While the invention has been described in detail, and with reference to specific embodiments thereof, it will be apparent to one of ordinary skill in the art that various changes and modifications can be made therein without departing from the spirit and scope of the invention.

### REFERENCES

Abhinandan and Martin, Mol Immunol. 45:3832-9, 2008
Adams et al., Acta Crystallogr D Biol Crystallogr. 66(Pt 2):213-21, 2010
Allen et al., J Neurosci. 22(21):9340-51, 2002
Almagro, Mol Recognit. 17:132-43, 2004
Asuni et al., J Neurosci. 27:9115-29, 2007
Boutajangout et al., J Neurochem. 118:658-67, 2011
Boutajangout et al., J Neurosci. 30:16559-66, 2010
Brunden et al., Nat Rev Drug Discov. 8:783-93, 2009
Butner and Kirschner, J Cell Biol. 115(3):717-30, 1991
Chai et al., J Biol Chem. 286:34457-67, 2011
Chothia and Lesk, J Mol Biol. 196:901-17, 1987
Clavaguera et al., Nat Cell Biol. 11:909-13, 2009
Clavaguera et al., Proc Natl Acad Sci U S A. 110(23):9535-40, 2013
Clavaguera et al., Proc Natl Acad Sci USA. 110(23):9535-40, 2013
Collaborative Computational Project, Number 4, Acta Crystallogr D Biol Crystallogr. 50(Pt 5):760-3, 1994
Collin et al., Brain. 137(Pt 10):2834-46, 2014
de Calignon et al., Neuron. 73(4):685-97, 2012
Dall'Acqua et al., J biol. Chem. 281(33); 23514-23524, 2006
Emsley and Cowtan, Acta Crystallogr D Biol Crystallogr. 60(Pt 12 Pt 1):2126-32, 2004
Epitope Mapping Protocols in Methods in Molecular Biology, Vol. 66, G. E. Morris, Ed. (1996)
Fishwild et al., Nat Biotechnol. 14:845-51, 1996
Fransson et al., J Mot Biol. 398(2):214-31, 2010
Frost et al., J Blol Chem. 284:12845-52, 2009
Funk et al., J Blol Chem. 290(35):21652-62, 2015
Galpern et al., Alzheimer's & Dementia: The Journal of the Alzheimer's Association 2019; 15(7) 252-253.
Goedert et al., Biochemical J. 301(Pt3):871-877
Hanger et al., Trends Mol Med. 15:112-9, 2009
Hoffmann et al., Biochemistry. 36(26):8114-24, 1997
Holmes et al., Proc Natl Acad Sci U S A. 111(41):E4376-85, 2014
Iba et al., Acta Neuropathol. 130(3):349-62, 2015
Iba et al., J Neurosci. 33(3):1024-37, 2013
Julien et al., Methods Mol Biol. 849:473-91, 2012
Kabsch, Acta Crystallogr D Biol Crystallogr. 66(Pt 2):125-32, 2010
Knappik et al., J Mol Biol. 296:57-86, 2000
Knight et al., Platelets. 15:409-18, 2004
Kohler and Milstein, Nature. 256:495-7, 1975
Krebs et al., J Immunol Methods. 254:67-84, 2001
Lee et al., Cell Rep. 16(6):1690-700, 2016
Lefranc et al., Dev Comp Immunol. 27:55-77, 2003
Leong et al., Cytokine. 16:106-19, 2001
Li and Lee, Biochemistry. 45(51):15692-701, 2006

Liu et al., Brain Imaging Behay. 6(4):610-20, 2012
Lonberg et al., Nature. 368:856-9, 1994
Malia et al., Proteins. 84:427-434, 2016
Martin and Thornton, J Mol Biol. 263(5):800-15, 1996
Matsuo et al., Neuron. 13(4):989-1002, 1994
McCoy et al., J Appl Crystallogr. 40(Pt 4):658-674, 2007
McEwan et al., 2017, PNAS 114(3):574-9
Mendez et al., Nat Genet. 15:146-56, 1997
Mercken et al., Acta Neuropathol. 84(3):265-72, 1992
Mercken, Ph.D. Thesis: University of Antwerp, Wilrijk-Antwerp, 1991
Mocanu et al., J Neurosci. 28(3):737-48, 2008
Morris et al., Nat Neurosci. 18(8):1183-9, 2015
Morris et al., Neuron, 70:410-26, 2011
Murshudov et al., Acta Crystallogr D Biol Crystallo 53(Pt 3):240-55, 1997
Oddo et al., J Neurochem. 102(4):1053-63, 2007
Otvos et al., J Neurosci Res. 39(6):669-73, 1994
Padlan et al., Mol. Immunol. 28:489-98, 1991
Peeraer et al., Neurobiol Dis. 73:83-95, 2015
Queen et al., Proc Natl Acad Sci USA. 86:10029-33, 1989
Scattoni et al., Behav Brain Res. 208(1):250-7, 2010
Schroeder et al., J Neuroimmune Pharmacol. 11(1):9-25, 2016
Seubert et al., J Biol Chem. 270(32):18917-22, 1995
Shi et al., J Mol Biol. 397:385-96, 2010
Strohl, Curr Opin Biotechnol. 20:685-91, 2009
Terwel et al., J Biol Chem. 280(5):3963-73, 2005
Vandermeeren et al., J Alzheimers Dis. 2018;65(1):265-281
Wischik et al. Proc Natl Acad Sci USA. 85:4884-8, 1988
Wu and Kabat, J Exp Med. 132:211-50, 1970
Yang et al., Protein Eng. 16:761-70, 2003
Yoshiyama et al., Neuron. 53(3):337-51, 2007
Zhao et al., Protein Expr Purif. 67(2):182-9, 2009

It is claimed:

1. An isolated monoclonal antibody or antigen-binding fragment thereof comprising a heavy chain variable region having a polypeptide sequence selected from SEQ ID NO: 71, 45, 51, 57, 63, 69, 39, 41, 43, 47, 49, 53, 55, 59, 61, 65, or 67, or a light chain variable region having a polypeptide sequence selected from SEQ ID NO: 72, 46, 52, 58, 64, 70, 40, 42, 44, 48, 50, 54, 56, 62, 66, or 68, wherein the monoclonal antibody or antigen-binding fragment thereof specifically binds paired helical filament (PHF)-tau, preferably human PHF-tau.

2. The isolated monoclonal antibody or antigen-binding fragment thereof of claim 1,
wherein the monoclonal antibody or antigen-binding fragment thereof comprises:
a. a heavy chain variable region having the polypeptide sequence of SEQ ID NO: 71, and a light chain variable region having the polypeptide sequence of SEQ ID NO: 72;
b. a heavy chain variable region having the polypeptide sequence of SEQ ID NO:45, and a light chain variable region having the polypeptide sequence of SEQ ID NO:46;
c. a heavy chain variable region having the polypeptide sequence of SEQ ID NO: 51, and a light chain variable region having the polypeptide sequence of SEQ ID NO: 52;
d. a heavy chain variable region having the polypeptide sequence of SEQ ID NO: 57, and a light chain variable region having the polypeptide sequence of SEQ ID NO: 58;
e. a heavy chain variable region having the polypeptide sequence of SEQ ID NO: 63, and a light chain variable region having the polypeptide sequence of SEQ ID NO: 64;
f. a heavy chain variable region having the polypeptide sequence of SEQ ID NO: 69, and a light chain variable region having the polypeptide sequence of SEQ ID NO: 70;
g. a heavy chain variable region having the polypeptide sequence of SEQ ID NO: 39, and a light chain variable region having the polypeptide sequence of SEQ ID NO: 40;
h. a heavy chain variable region having the polypeptide sequence of SEQ ID NO: 41, and a light chain variable region having the polypeptide sequence of SEQ ID NO: 42;
i. a heavy chain variable region having the polypeptide sequence of SEQ ID NO: 43, and a light chain variable region having the polypeptide sequence of SEQ ID NO: 44;
j. a heavy chain variable region having the polypeptide sequence of SEQ ID NO: 47, and a light chain variable region having the polypeptide sequence of SEQ ID NO: 48;
k. a heavy chain variable region having the polypeptide sequence of SEQ ID NO: 49, and a light chain variable region having the polypeptide sequence of SEQ ID NO: 50;
l. a heavy chain variable region having the polypeptide sequence of SEQ ID NO: 53, and a light chain variable region having the polypeptide sequence of SEQ ID NO: 54;
m. a heavy chain variable region having the polypeptide sequence of SEQ ID NO: 55, and a light chain variable region having the polypeptide sequence of SEQ ID NO: 56;
n. a heavy chain variable region having the polypeptide sequence of SEQ ID NO: 59, and a light chain variable region having the polypeptide sequence of SEQ ID NO: 60;
o. a heavy chain variable region having the polypeptide sequence of SEQ ID NO: 61, and a light chain variable region having the polypeptide sequence of SEQ ID NO: 62;
p. a heavy chain variable region having the polypeptide sequence of SEQ ID NO: 65, and a light chain variable region having the polypeptide sequence of SEQ ID NO: 66; or
q. a heavy chain variable region having the polypeptide sequence of SEQ ID NO: 67, and a light chain variable region having the polypeptide sequence of SEQ ID NO: 68.

3. The isolated monoclonal antibody or antigen-binding fragment thereof of claim 1,
wherein the monoclonal antibody or antigen-binding fragment thereof comprises:
a. a heavy chain having the polypeptide sequence of SEQ ID NO: 37, and a light chain having the polypeptide sequence of SEQ ID NO: 38;
b. a heavy chain having the polypeptide sequence of SEQ ID NO: 11, and a light chain having the polypeptide sequence of SEQ ID NO: 12;
c. a heavy chain having the polypeptide sequence of SEQ ID NO: 17, and a light chain having the polypeptide sequence of SEQ ID NO: 18;
d. a heavy chain having the polypeptide sequence of SEQ ID NO: 23, and a light chain having the polypeptide sequence of SEQ ID NO: 24;
e. a heavy chain having the polypeptide sequence of SEQ ID NO: 29, and a light chain having the polypeptide sequence of SEQ ID NO: 30;
f. a heavy chain having the polypeptide sequence of SEQ ID NO: 35, and a light chain having the polypeptide sequence of SEQ ID NO: 36;
g. a heavy chain having the polypeptide sequence of SEQ ID NO: 5, and a light chain having the polypeptide sequence of SEQ ID NO: 6;
h. a heavy chain having the polypeptide sequence of SEQ ID NO: 7, and a light chain having the polypeptide sequence of SEQ ID NO: 8;
i. a heavy chain having the polypeptide sequence of SEQ ID NO: 9, and a light chain having the polypeptide sequence of SEQ ID NO: 10;
j. a heavy chain having the polypeptide sequence of SEQ ID NO: 13, and a light chain having the polypeptide sequence of SEQ ID NO: 14;
k. a heavy chain having the polypeptide sequence of SEQ ID NO: 15, and a light chain having the polypeptide sequence of SEQ ID NO: 16;
l. a heavy chain having the polypeptide sequence of SEQ ID NO: 19, and a light chain having the polypeptide sequence of SEQ ID NO: 20;
m. a heavy chain having the polypeptide sequence of SEQ ID NO: 21, and a light chain having the polypeptide sequence of SEQ ID NO: 22;
n. a heavy chain having the polypeptide sequence of SEQ ID NO: 25, and a light chain having the polypeptide sequence of SEQ ID NO: 26;
o. a heavy chain having the polypeptide sequence of SEQ ID NO: 27, and a light chain having the polypeptide sequence of SEQ ID NO: 28;
p. a heavy chain having the polypeptide sequence of SEQ ID NO: 31, and a light chain having the polypeptide sequence of SEQ ID NO: 32; or
q. a heavy chain having the polypeptide sequence of SEQ ID NO: 33, and a light chain having the polypeptide sequence of SEQ ID NO: 34.

4. An isolated nucleic acid encoding the monoclonal antibody or antigen-binding fragment thereof of any one of claims 1 to 3.

5. A vector comprising the isolated nucleic acid of claim 4.

6. A host cell comprising the nucleic acid of claim 5.

7. A pharmaceutical composition comprising the isolated monoclonal antibody or antigen-binding fragment thereof of any one of claims 1 to 3 and a pharmaceutically acceptable carrier.

8. A method of reducing pathological tau aggregation or spreading of tauopathy in a subject in need thereof, comprising administering to the subject the pharmaceutical composition of claim 7.

9. A method of treating a tauopathy in a subject in need thereof, comprising administering to the subject the pharmaceutical composition of claim 7.

10. The method of claim 9 wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

11. A method of producing a monoclonal antibody or antigen-binding fragment thereof of any one of claims 1 to 3, the method comprising culturing a cell comprising a nucleic acid encoding the monoclonal antibody or antigen-binding fragment thereof under conditions to produce the monoclonal antibody or antigen-binding fragment thereof and recovering the monoclonal antibody or antigen-binding fragment thereof from the cell or cell culture.

12. A method of detecting the presence of PHF-tau in a biological sample from a subject, comprising contacting the biological sample with the monoclonal antibody or antigen-binding fragment thereof of any one of claims 1 to 3 and detecting binding of the monoclonal antibody or antigen-binding fragment thereof to PHF-tau in the sample from the subject.

13. The method of claim 12, wherein the biological sample is a blood, serum, plasma, interstitial fluid, or cerebral spinal fluid sample.


SEQUENCE LISTING (84 sequences)

SEQ ID NO: 1 - protein, 118 aa, Artificial Sequence
  Description of Artificial Sequence Synthetic polypeptide
  PT3 HC
EVKLVESGGDLVKPGGSLKLSCAASGFTFSSYAMSWVRQNPEKRLEWVASISKGGNTYYPNSVKGRFTISRDNARNILYLQMSSLRSEDTALYYCARGWGDYGWFAYWGQVTLVTVSA

SEQ ID NO: 2 - protein, 107 aa, Artificial Sequence
  Description of Artificial Sequence Synthetic polypeptide
  PT3 LC
DIKMTQSPSSMYASLGERVTITCKASQDINRYLNWFQQKPGKSPKTLIYRANRLLDGVPSRFSGSGSGQDYSLTISSLDYEDMGIYYCLQYDEFPLTFGDGTKLELK

SEQ ID NO: 3 - protein, 118 aa, Artificial Sequence
  Description of Artificial Sequence Synthetic polypeptide
  Graft HC
EVQLVESGGGLVKPGGSLRLSCAASGFTFSSYAMSWVRQAPGKGLEWVSSISKGGNTYYADSVKGRFTISRDNAKNSLYLQMNSLRAEDTAVYYCARGWGDYGWFAYWGQVTLVTVSS

SEQ ID NO: 4 - protein, 107 aa, Artificial Sequence
  Description of Artificial Sequence Synthetic polypeptide
  Graft LC
DIQMTQSPSSLSASVGDRVTITCKASQDINRYLNWFQQKPGKAPKSLIYRANRLLDGVPSRFSGSGSGTDFTLTISSLQPEDFATYYCLQYDEFPLTFGQGTKLEIK

SEQ ID NO: 5 - protein, 448 aa, Artificial Sequence
  Description of Artificial Sequence Synthetic polypeptide
  PT1B841 HC
EVQLVESGGGLVKPGGSLRLSCAASGFTFSSYAMSWVRQAPGKGLEWVSSISKGGNTYYADSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Description of Artificial Sequence Synthetic polypeptide
  PT1B841 LC
DIQMTQSPSSLSASVGDRVTITCKASQDINRYLNWFQQKPGKAPKSLIYRANRLLDGVPSRFSGSGSGTDFTLTISSLQPEDFATYYCLQYDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 448 aa, Artificial Sequence
  Description of Artificial Sequence Synthetic polypeptide
  PT1B842 HC
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, Artificial Sequence
  Description of Artificial Sequence Synthetic polypeptide
  PT1B842 LC
DIQMTQSPSSLSASVGDRVTITCKASQDINRYLNWFQQKPGKAPKSLIYRANRLLSGVPSRFSGSGSGTDFTLTISSLQPEDMATYYCQQYDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 448 aa, Artificial Sequence; identical to SEQ ID NO: 7
  Description of Artificial Sequence Synthetic polypeptide
  PT1B843 HC

SEQ ID NO: 10 - protein, 214 aa, Artificial Sequence
  Description of Artificial Sequence Synthetic polypeptide
  PT1B843 LC
DIQMTQSPSSLSASVGDRVTITCKASQDINRYLNWFQQKPGKSPKTLIYRANRLLSGVPSRFSGSGSGTDFTLTISSLQPEDFATYYCQQYDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 448 aa, Artificial Sequence; identical to SEQ ID NO: 7
  Description of Artificial Sequence Synthetic polypeptide
  PT1B844 HC

SEQ ID NO: 12 - protein, 214 aa, Artificial Sequence
  Description of Artificial Sequence Synthetic polypeptide
  PT1B844 LC
DIQMTQSPSSLSASVGDRVTITCKASQDINRYLNWFQQKPGKAPKTLIYRANRLLDGVPSRFSGSGSGTDFTLTISSLQPEDFATYYCQQYDEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 448 aa, Artificial Sequence
  Description of Artificial Sequence Synthetic polypeptide
  PT1B845 HC
EVQLVESGGGLVKPGGSLRLSCAASGFTFSSYAMSWVRQAPGKRLEWVASISKGGNTYYPNSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, Artificial Sequence; identical to SEQ ID NO: 8
  Description of Artificial Sequence Synthetic polypeptide
  PT1B845 LC

SEQ ID NO: 15 - protein, 448 aa, Artificial Sequence; identical to SEQ ID NO: 13
  Description of Artificial Sequence Synthetic polypeptide
  PT1B846 HC

SEQ ID NO: 16 - protein, 214 aa, Artificial Sequence; identical to SEQ ID NO: 10
  Description of Artificial Sequence Synthetic polypeptide
  PT1B846 LC

SEQ ID NO: 17 - protein, 448 aa, Artificial Sequence; identical to SEQ ID NO: 13
  Description of Artificial Sequence Synthetic polypeptide
  PT1B847 HC

SEQ ID NO: 18 - protein, 214 aa, Artificial Sequence; identical to SEQ ID NO: 12
  Description of Artificial Sequence Synthetic polypeptide
  PT1B847 LC

SEQ ID NO: 19 - protein, 448 aa, Artificial Sequence
  Description of Artificial Sequence Synthetic polypeptide
  PT1B848 HC
EVQLVESGGGLVKPGGSLRLSCAASGFTFSSYAMSWVRQAPGKRLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, Artificial Sequence; identical to SEQ ID NO: 8
  Description of Artificial Sequence Synthetic polypeptide
  PT1B848 LC

SEQ ID NO: 21 - protein, 448 aa, Artificial Sequence; identical to SEQ ID NO: 19
  Description of Artificial Sequence Synthetic polypeptide
  PT1B849 HC

SEQ ID NO: 22 - protein, 214 aa, Artificial Sequence; identical to SEQ ID NO: 10
  Description of Artificial Sequence Synthetic polypeptide
  PT1B849 LC

SEQ ID NO: 23 - protein, 448 aa, Artificial Sequence; identical to SEQ ID NO: 19
  Description of Artificial Sequence Synthetic polypeptide
  PT1B850 HC

SEQ ID NO: 24 - protein, 214 aa, Artificial Sequence; identical to SEQ ID NO: 12
  Description of Artificial Sequence Synthetic polypeptide
  PT1B850 LC

SEQ ID NO: 25 - protein, 448 aa, Artificial Sequence
  Description of Artificial Sequence Synthetic polypeptide
  PT1B851 HC
EVQLVESGGGLVKPGGSLRLSCAASGFTFSSYAMSWVRQAPGKGLEWVASISKGGNTYYPNSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, Artificial Sequence; identical to SEQ ID NO: 8
  Description of Artificial Sequence Synthetic polypeptide
  PT1B851 LC

SEQ ID NO: 27 - protein, 448 aa, Artificial Sequence; identical to SEQ ID NO: 25
  Description of Artificial Sequence Synthetic polypeptide
  PT1B852 HC

SEQ ID NO: 28 - protein, 214 aa, Artificial Sequence; identical to SEQ ID NO: 10
  Description of Artificial Sequence Synthetic polypeptide
  PT1B852 LC

SEQ ID NO: 29 - protein, 448 aa, Artificial Sequence; identical to SEQ ID NO: 25
  Description of Artificial Sequence Synthetic polypeptide
  PT1B853 HC

SEQ ID NO: 30 - protein, 214 aa, Artificial Sequence; identical to SEQ ID NO: 12
  Description of Artificial Sequence Synthetic polypeptide
  PT1B853 LC

SEQ ID NO: 31 - protein, 448 aa, Artificial Sequence
  Description of Artificial Sequence Synthetic polypeptide
  PT1B854 HC
EVQLVESGGGLVKPGGSLRLSCAASGFTFSSYAMSWVRQNPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 214 aa, Artificial Sequence; identical to SEQ ID NO: 8
  Description of Artificial Sequence Synthetic polypeptide
  PT1B854 LC

SEQ ID NO: 33 - protein, 448 aa, Artificial Sequence; identical to SEQ ID NO: 31
  Description of Artificial Sequence Synthetic polypeptide
  PT1B855 HC

SEQ ID NO: 34 - protein, 214 aa, Artificial Sequence; identical to SEQ ID NO: 10
  Description of Artificial Sequence Synthetic polypeptide
  PT1B855 LC

SEQ ID NO: 35 - protein, 448 aa, Artificial Sequence; identical to SEQ ID NO: 31
  Description of Artificial Sequence Synthetic polypeptide
  PT1B856 HC

SEQ ID NO: 36 - protein, 214 aa, Artificial Sequence; identical to SEQ ID NO: 12
  Description of Artificial Sequence Synthetic polypeptide
  PT1B856 LC

SEQ ID NO: 37 - protein, 448 aa, Artificial Sequence
  Description of Artificial Sequence Synthetic polypeptide
  PT1B916 HC
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, Artificial Sequence; identical to SEQ ID NO: 12
  Description of Artificial Sequence Synthetic polypeptide
  PT1B916 LC

SEQ ID NO: 39 - protein, 118 aa, Artificial Sequence; identical to SEQ ID NO: 3
  Description of Artificial Sequence Synthetic polypeptide
  PT1B841 VH

SEQ ID NO: 40 - protein, 107 aa, Artificial Sequence; identical to SEQ ID NO: 4
  Description of Artificial Sequence Synthetic polypeptide
  PT1B841 VL

SEQ ID NO: 41 - protein, 118 aa, Artificial Sequence
  Description of Artificial Sequence Synthetic polypeptide
  PT1B842 VH
EVQLVESGGGLVKPGGSLRLSCAASGFTFSSYAMSWVRQAPGKGLEWVASISKGGNTYYPDSVKGRFTISRDNAKNSLYLQMNSLRAEDTAVYYCARGWGDYGWFAYWGQVTLVTVSS

SEQ ID NO: 42 - protein, 107 aa, Artificial Sequence
  Description of Artificial Sequence Synthetic polypeptide
  PT1B842 VL
DIQMTQSPSSLSASVGDRVTITCKASQDINRYLNWFQQKPGKAPKSLIYRANRLLSGVPSRFSGSGSGTDFTLTISSLQPEDMATYYCQQYDEFPLTFGQGTKLEIK

SEQ ID NO: 43 - protein, 118 aa, Artificial Sequence; identical to SEQ ID NO: 41
  Description of Artificial Sequence Synthetic polypeptide
  PT1B843 VH

SEQ ID NO: 44 - protein, 107 aa, Artificial Sequence
  Description of Artificial Sequence Synthetic polypeptide
  PT1B843 VL
DIQMTQSPSSLSASVGDRVTITCKASQDINRYLNWFQQKPGKSPKTLIYRANRLLSGVPSRFSGSGSGTDFTLTISSLQPEDFATYYCQQYDEFPLTFGQGTKLEIK

SEQ ID NO: 45 - protein, 118 aa, Artificial Sequence; identical to SEQ ID NO: 41
  Description of Artificial Sequence Synthetic polypeptide
  PT1B844 VH

SEQ ID NO: 46 - protein, 107 aa, Artificial Sequence
  Description of Artificial Sequence Synthetic polypeptide
  PT1B844 VL
DIQMTQSPSSLSASVGDRVTITCKASQDINRYLNWFQQKPGKAPKTLIYRANRLLDGVPSRFSGSGSGTDFTLTISSLQPEDFATYYCQQYDEFPLTFGQGTKLEIK

SEQ ID NO: 47 - protein, 118 aa, Artificial Sequence
  Description of Artificial Sequence Synthetic polypeptide
  PT1B845 VH
EVQLVESGGGLVKPGGSLRLSCAASGFTFSSYAMSWVRQAPGKRLEWVASISKGGNTYYPNSVKGRFTISRDNAKNSLYLQMNSLRAEDTAVYYCARGWGDYGWFAYWGQVTLVTVSS

SEQ ID NO: 48 - protein, 107 aa, Artificial Sequence; identical to SEQ ID NO: 42
  Description of Artificial Sequence Synthetic polypeptide
  PT1B845 VL

SEQ ID NO: 49 - protein, 118 aa, Artificial Sequence; identical to SEQ ID NO: 47
  Description of Artificial Sequence Synthetic polypeptide
  PT1B846 VH

SEQ ID NO: 50 - protein, 107 aa, Artificial Sequence; identical to SEQ ID NO: 44
  Description of Artificial Sequence Synthetic polypeptide
  PT1B846 VL

SEQ ID NO: 51 - protein, 118 aa, Artificial Sequence; identical to SEQ ID NO: 47
  Description of Artificial Sequence Synthetic polypeptide
  PT1B847 VH

SEQ ID NO: 52 - protein, 107 aa, Artificial Sequence; identical to SEQ ID NO: 46
  Description of Artificial Sequence Synthetic polypeptide
  PT1B847 VL

SEQ ID NO: 53 - protein, 118 aa, Artificial Sequence
  Description of Artificial Sequence Synthetic polypeptide
  PT1B848 VH
EVQLVESGGGLVKPGGSLRLSCAASGFTFSSYAMSWVRQAPGKRLEWVASISKGGNTYYPDSVKGRFTISRDNAKNSLYLQMNSLRAEDTAVYYCARGWGDYGWFAYWGQVTLVTVSS

SEQ ID NO: 54 - protein, 107 aa, Artificial Sequence; identical to SEQ ID NO: 42
  Description of Artificial Sequence Synthetic polypeptide
  PT1B848 VL

SEQ ID NO: 55 - protein, 118 aa, Artificial Sequence; identical to SEQ ID NO: 53
  Description of Artificial Sequence Synthetic polypeptide
  PT1B849 VH

SEQ ID NO: 56 - protein, 107 aa, Artificial Sequence; identical to SEQ ID NO: 44
  Description of Artificial Sequence Synthetic polypeptide
  PT1B849 VL

SEQ ID NO: 57 - protein, 118 aa, Artificial Sequence; identical to SEQ ID NO: 53
  Description of Artificial Sequence Synthetic polypeptide
  PT1B850 VH

SEQ ID NO: 58 - protein, 107 aa, Artificial Sequence; identical to SEQ ID NO: 46
  Description of Artificial Sequence Synthetic polypeptide
  PT1B850 VL

SEQ ID NO: 59 - protein, 118 aa, Artificial Sequence
  Description of Artificial Sequence Synthetic polypeptide
  PT1B851 VH
EVQLVESGGGLVKPGGSLRLSCAASGFTFSSYAMSWVRQAPGKGLEWVASISKGGNTYYPNSVKGRFTISRDNAKNSLYLQMNSLRAEDTAVYYCARGWGDYGWFAYWGQVTLVTVSS

SEQ ID NO: 60 - protein, 107 aa, Artificial Sequence; identical to SEQ ID NO: 42
  Description of Artificial Sequence Synthetic polypeptide
  PT1B851 VL

SEQ ID NO: 61 - protein, 118 aa, Artificial Sequence; identical to SEQ ID NO: 59
  Description of Artificial Sequence Synthetic polypeptide
  PT1B852 VH

SEQ ID NO: 62 - protein, 107 aa, Artificial Sequence; identical to SEQ ID NO: 44
  Description of Artificial Sequence Synthetic polypeptide
  PT1B852 VL

SEQ ID NO: 63 - protein, 118 aa, Artificial Sequence; identical to SEQ ID NO: 59
  Description of Artificial Sequence Synthetic polypeptide
  PT1B853 VH

SEQ ID NO: 64 - protein, 107 aa, Artificial Sequence; identical to SEQ ID NO: 46
  Description of Artificial Sequence Synthetic polypeptide
  PT1B853 VL

SEQ ID NO: 65 - protein, 118 aa, Artificial Sequence
  Description of Artificial Sequence Synthetic polypeptide
  PT1B854 VH
EVQLVESGGGLVKPGGSLRLSCAASGFTFSSYAMSWVRQNPGKGLEWVASISKGGNTYYPDSVKGRFTISRDNAKNSLYLQMNSLRAEDTAVYYCARGWGDYGWFAYWGQVTLVTVSS

SEQ ID NO: 66 - protein, 107 aa, Artificial Sequence; identical to SEQ ID NO: 42
  Description of Artificial Sequence Synthetic polypeptide
  PT1B854 VL

SEQ ID NO: 67 - protein, 118 aa, Artificial Sequence; identical to SEQ ID NO: 65
  Description of Artificial Sequence Synthetic polypeptide
  PT1B855 VH

SEQ ID NO: 68 - protein, 107 aa, Artificial Sequence; identical to SEQ ID NO: 44
  Description of Artificial Sequence Synthetic polypeptide
  PT1B855 VL

SEQ ID NO: 69 - protein, 118 aa, Artificial Sequence; identical to SEQ ID NO: 65
  Description of Artificial Sequence Synthetic polypeptide
  PT1B856 VH

SEQ ID NO: 70 - protein, 107 aa, Artificial Sequence; identical to SEQ ID NO: 46
  Description of Artificial Sequence Synthetic polypeptide
  PT1B856 VL

SEQ ID NO: 71 - protein, 118 aa, Artificial Sequence; identical to SEQ ID NO: 41
  Description of Artificial Sequence Synthetic polypeptide
  PT1B916 VH

SEQ ID NO: 72 - protein, 107 aa, Artificial Sequence; identical to SEQ ID NO: 46
  Description of Artificial Sequence Synthetic polypeptide
  PT1B916 VL

SEQ ID NO: 73 - protein, 352 aa, Artificial Sequence
  Description of Artificial Sequence Synthetic polypeptide
  Tau 0N3R
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 74 - protein, 381 aa, Artificial Sequence
  Description of Artificial Sequence Synthetic polypeptide
  Tau 1N3R
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 75 - protein, 410 aa, Artificial Sequence
  Description of Artificial Sequence Synthetic polypeptide
  Tau 2N3R
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 76 - protein, 383 aa, Artificial Sequence
  Description of Artificial Sequence Synthetic polypeptide
  Tau 0N4R
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 77 - protein, 441 aa, Artificial Sequence
  Description of Artificial Sequence Synthetic polypeptide
  Tau 1N4R
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 78 - protein, 441 aa, Artificial Sequence; identical to SEQ ID NO: 77
  Description of Artificial Sequence Synthetic polypeptide
  Tau 2N4R

SEQ ID NO: 79 - protein, 58 aa, Artificial Sequence
  Description of Artificial Sequence Synthetic polypeptide
  Tau calibrant peptide 1
  MOD_RES 44)..(44): Phosphorylated Thr
  MOD_RES (49)..(49): Phosphorylated Thr
  MOD_RES (58)..(58): Amidated Val
PRQEFEVMEDHAGTYGLGDRGKTKIATPRGAAPPGQKGGSRSRTPSLPTPPTREPKKV

SEQ ID NO: 80 - protein, 38 aa, Artificial Sequence
  Description of Artificial Sequence Synthetic polypeptide
  Tau calibrant peptide 2
  MOD_RES (1)..(1): Acetylated Ser
  MOD_RES (24)..(24): Phosphorylated Thr
  MOD_RES (29)..(29): Phosphorylated Thr
  MOD_RES (38)..(38): Amidated Val
SLEDEAAGHVTQARMVSKGSRSRTPSLPTPPTREPKKV

SEQ ID NO: 81 - protein, 20 aa, Artificial Sequence
  Description of Artificial Sequence Synthetic peptide
PRQEFEVMEDHAGTYGLGDR

SEQ ID NO: 82 - protein, 18 aa, Artificial Sequence
  Description of Artificial Sequence Synthetic peptide
GKTKIATPRGAAPPGQKG

SEQ ID NO: 83 - protein, 20 aa, Artificial Sequence
  Description of Artificial Sequence Synthetic peptide
  MOD_RES (6)..(6): Phosphorylated Thr
  MOD_RES (11)..(11): Phosphorylated Thr
  MOD_RES (20)..(20): Amidated Val
GSRSRTPSLPTPPTREPKKV

SEQ ID NO: 84 - protein, 18 aa, Artificial Sequence
  Description of Artificial Sequence Synthetic peptide
SLEDEAAGHVTQARMVSK